(19) United States
(12) Patent Application Publication
(10) Pub. No.: US 2014/0349297 A1
(43) Pub. Date: Nov. 27, 2014

(54) FLUORESCENT DYES BASED ON ACRIDINE AND ACRIDINIUM DERIVATIVES

(71) Applicant: Almac Sciences (Scotland) Limited, Gladsmuir (GB)

(72) Inventors: Beatrice Maltman, East Lothian (GB); Adina-Elena Tirnaveanu, East Lothian (GB); Graham Cotton, East Lothian (GB)

(73) Assignee: Almac Sciences (Scotland) Limited, Gladsmuir (GB)

(21) Appl. No.: 14/367,573

(22) PCT Filed: Dec. 20, 2012
(86) PCT No.: PCT/GB2012/053214

§ 371 Date: Jun. 20, 2014

(30) Foreign Application Priority Data
Dec. 22, 2011 (GB) 1122099.3

(51) Int. Cl. G01N 33/53; G01N 33/52

(52) U.S. Cl. CPC G01N 33/5306; G01N 33/52; G01N 2458/00
USPC 435/6.11; 435/15; 546/102; 435/23; 435/7.1

(57) ABSTRACT

The present invention relates to fluorescent dyes based on acridine and acridinium derivatives and use of such dyes in for example, biochemical and/or cell-based assays.

### FIELD

The present invention relates to fluorescent dyes based on acridine and acridinium derivatives and use of such dyes in, for example, biochemical and/or cell-based assays.

### BACKGROUND

Fluorescent molecules, including dyes, have long been used as agents for labelling and detecting biological molecules in cell-free biochemical assays, as well as cell-based assays. However, in many systems, there is background fluorescence and it is necessary to have a good signal-to-noise ratio in order to successfully detect the relevant fluorescent signal.

Many highly fluorescent dyes are known and used in order to improve signal-to-noise ratio. An alternative way of addressing the issue of background fluorescence is to use fluorescent molecules that display a fluorescence lifetime different to the system being studied. By doing so, detection of the fluorescent molecule can be discerned from background.

Previously, we have described the use of 9-aminoacridine derivatives as long lifetime fluorescence reporters in bioassays (see WO 2007/049057 A2; G. Cotton et al., Chem. Commun., 2010, 46, 6929; and A. Gray et al., Anal. Biochem., 2010, 402, 54). However, there is an ever-present need for new fluorescent molecules and/or identification of known molecules that have useful fluorescent properties, for example fluorescence lifetimes, for use in biochemical and cell based assays.

### SUMMARY

We have surprisingly found that a series of fluorophores based on acridine and acridinium derivatives in which no amino group is attached at the 9-position, but rather a group of predominantly hydrocarbyl character is present, are suitable for use in biochemical and cell based assays. This finding is particularly surprising given the knowledge in the art of the strong fluorescence of 9-aminoacridine and its derivatives.

Viewed from a first aspect, therefore, the invention provides the use of a fluorescent dye of formula (I):

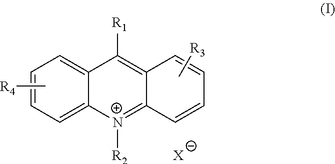

(wherein:

R¹ is hydrogen or J-L;

R² is absent, hydrogen or J-L;

R³ and R⁴ are independently at each occurrence selected from hydrogen, halo, amido, hydroxyl, substituted or unsubstituted alkyl, substituted or unsubstituted alkenyl, substituted or unsubstituted aryl, alkoxy, alkylthio, amino, mono- or di-C₁-C₄ alkyl-substituted amino, sulfhydryl, carboxy, acyl, formyl, sulfonate, quaternary ammonium, J-L, or —K;

X is absent if R² is absent, and if R² is present the nitrogen atom to which it is attached is positively charged and X is a counter ion;

each J is independently a linker group;

each L is independently hydrogen or K; and

each K is independently a target bonding group, provided that at least one group K is present) as a reagent in a method to detect a target molecule, the method comprising the measurement of lifetime fluorescence.

Viewed from a second aspect, the invention provides a method for determining the presence of an analyte in a sample, which method comprises:

(i) contacting the sample with a conjugate of a known binding partner of the analyte and a fluorescent dye of the formula (I):

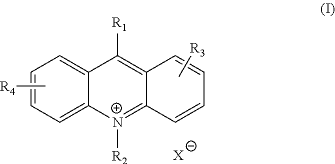

(wherein:

R¹ is hydrogen or J-L;

R² is absent, hydrogen or J-L;

R³ and R⁴ are independently at each occurrence selected from hydrogen, halo, amido, hydroxyl, substituted or unsubstituted alkyl, substituted or unsubstituted alkenyl, substituted or unsubstituted aryl, alkoxy, alkylthio, amino, mono- or di-C₁-C₄ alkyl-substituted amino, sulfhydryl, carboxy, acyl, formyl, sulfonate, quaternary ammonium, J-L, or —K;

X is a counter ion, which is absent if R² is absent;

each J is independently a linker group;

each L is independently hydrogen or K; and

each K is independently a target bonding group, provided that at least one group K is present) under conditions effective to allow binding of at least a portion of the analyte to the known binding partner within the conjugate to form a complex of the analyte and the conjugate;

(ii) measuring the fluorescence lifetime or fluorescence intensity of the conjugate before contact with the analyte; and

(iv) measuring the fluorescent lifetime or fluorescence intensity of the mixture resultant from the contacting.

Viewed from a third aspect, the invention provides a method of measuring activity of an enzyme in the presence of a conjugate, which is a conjugate resultant from conjugation between a compound and a fluorescent dye of the formula (I):

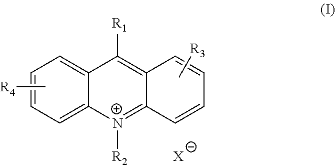

(wherein:

R¹ is hydrogen or J-L;

R² is absent, hydrogen or J-L;

R³ and R⁴ are independently at each occurrence selected from hydrogen, halo, amido, hydroxyl, substituted or unsubstituted alkyl, substituted or unsubstituted alkenyl, substituted or unsubstituted aryl, alkoxy, alkylthio, amino, mono- or di-C₁-C₄ alkyl-substituted amino, sulfhydryl, acyl, formyl, carboxy, sulfonate, quaternary ammonium, J-L, or —K;

X is a counter ion, which is absent if R² is absent;

each J is independently a linker group;

each L is independently hydrogen or K; and

each K is independently a target bonding group, provided that at least one group K is present),

which method comprises:

(i) measuring the fluorescence lifetime or fluorescence intensity of the conjugate before contact with the enzyme;

(ii) contacting the enzyme with the conjugate; and

(iii) measuring the fluorescent lifetime or fluorescence intensity of the mixture resultant from the contacting.

Viewed from a fourth aspect, the invention provides a fluorescent dye of the formula (I) as hereinbefore defined wherein:

R¹ is hydrogen or J-H;

R² is J-L; and

R³ is hydrogen or J-K.

Viewed from a fifth aspect, the invention provides a conjugate of a fluorescent dye according to the fourth aspect of the invention and a biological molecule.

Viewed from a sixth aspect, the invention provides a kit comprising:

(i) a conjugate in accordance with the fifth aspect of the invention; and

(ii) a known binding partner of the biological molecule, for example an enzyme.

Further aspects and embodiments of the present invention will be evident from the discussion that follows below.

### BRIEF DESCRIPTION OF THE FIGURES

FIG. 1 shows fluorescence properties of 9,10-dimethylacridin-10-ium methyl sulfate (a) fluorescence excitation and emission spectra (b) fluorescence lifetime decay curve (26.2 ns). Measurements were performed in 10 mM PBS pH 7.4, excitation wavelength 350 nm and emission wavelength 490 nm for steady state, and excitation wavelength 405 nm and emission wavelength 473 nm long pass filter for fluorescence lifetime;

FIG. 2 shows the fluorescence lifetime of 9,10-dimethylacridin-10-ium methyl sulfate in phosphate buffer measured as a function of pH. 1 μM solutions of 9,10-dimethylacridin-10-ium methyl sulfate in 20 mM sodium phosphate buffer were prepared using 0.2 M monobasic sodium phosphate and 0.2 M dibasic sodium phosphate buffer mixtures, 0.1 M HCl solution was used to adust the pH to <6 and 0.1 M NaOH solution was used to adjust the pH to >8;

FIG. 3 shows the fluorescence emission spectra for 9,10-dimethylacridin-10-ium methyl sulfate measured as a function of pH upon excitation at 405 nm;

FIG. 4 shows fluorescence emission spectra for LLD-DEVDSK and LLD-DEVDSW (LLD=with 2-(2-carboxyethyl)-9,10-dimethylacridin-10-ium). Measurements were performed at 500 nM concentration in 10 mM PBS pH 7.4, excitation wavelength 405 nm. Solid line is LLD-DEVDSK and dashed line is LLD-DEVDSW;

FIG. 5 shows a plot of average lifetime against time for a Caspase 3 assay using LLD-DEVDSW as substrate and recombinant Caspase 3 enzyme (1.25 and 2.5 U per well).

FIG. 6 shows Caspase 3 inhibition by AcDEVD-CHO, viz an inhibitor titration of AcDEVD-CHO against recombinant Caspase 3 using LLD-DEVDSW as substrate.

FIG. 7 shows a plot of average lifetime against time for a MMP2 protease assay using LLD-PLGLNaIAR as substrate and recombinant MMP2 enzyme with different concentrations of enzyme.

FIG. 8 shows a plot of average lifetime against time for a Lck kinase assay using LLD-EPEGIYGVLF as substrate and recombinant Lck enzyme with different concentrations of enzyme.

FIG. 9 shows an inhibitor titration of staurosporine against recombinant Lck kinase using LLD-EPEGIYGVLF as substrate.

FIG. 10 shows a titration of various concentrations of ATP against recombinant Lck kinase using LLD-EPEGIYGVLF as substrate, so as to determine the ATP K, for the system assayed.

### DETAILED DESCRIPTION

The present invention arises from the finding that fluorophores based on acridine and acridinium derivatives in which no amino group is attached at the 9-position, but rather a group of predominantly hydrocarbyl character is present at this position, are suitable for use in biochemical and cell-based assays. In particular, some of the fluorophores of and/or uses according to the various aspects of the invention have advantageously long fluorescence lifetimes (for example 25 to 30 ns). These may be contrasted favourably with 9-aminoacridines, which typically have fluorescence lifetimes of approximately 15 to 17 ns.

As is known in the art, a longer fluorescence lifetime can be used to improve the signal-to-noise ratio, allowing the potential for more sensitive response in assays. Unlike fluorescence intensity, fluorescence lifetime is generally independent of probe concentration and volume, and unaffected by auto-fluorescence, light scattering and inner filter effects. Additionally, measurement of fluorescence lifetime enables background interference from fluorescent compound libraries and cellular components to be minimised, affording less false positives in drug screening applications. Accordingly, it is typical, but not necessarily, to measure fluorescence lifetime (as opposed to fluorescence intensity) according to the second and third aspects of the invention.

Firstly, the compounds of formula (I) are described, with the following definitions applying unless the context dictates to the contrary.

By alkyl is meant herein a saturated hydrocarbyl radical, which may be straight-chain, cyclic or branched (typically straight-chain unless the context dictates to the contrary). An alkylene group is a diradical formed formally by abstraction of a hydrogen atom from an alkyl group. Typically alkyl and alkylene groups comprise from 1 to 25 carbon atoms, more usually 1 to 10 carbon atoms, more usually still 1 to 6 carbon atoms, it being of course understood that the lower limit to the number of carbon atoms in cycloalkyl and cycloalkylene groups is 3.

Alkenyl and alkynyl groups differ from alkyl groups in having one or more sites of unsaturation, constituted by carbon-carbon double bonds or carbon-carbon triple bonds. The presence of a carbon-carbon double bond provides an alkenyl group; the presence of a carbon-carbon triple bond provides an alkynyl group. Alkenylene and alkynylene groups are diradicals formed formally by abstraction of a hydrogen atom from alkenyl and alkynyl groups respectively. Typically, alkenyl, alkenylene, alkynyl and alkynylene groups comprise from 2 to 25 carbon atoms, more usually 2 to 10 carbon atoms, more usually still 2 to 6 carbon atoms. Examples of alkenyl groups include vinyl, styryl and acrylate; an example of an alkynyl group is propargyl. For the avoidance of any doubt, a hydrocarbyl radical comprising both a carbon-carbon double bond and a carbon-carbon triple bond may be regarded as both an alkenyl and an alkynyl group.

Alkyl, alkenyl or alkynyl (and alkylene, alkenylene and alkynylene) groups may be substituted, for example once, twice, or three times, e.g. once, i.e. formally replacing one or more hydrogen atoms of the group. Examples of such substituents are hydroxy, amino, halo, aryl, (including heteroaryl), nitro, alkoxy, alkylthio, cyano, sulfhydryl, acyl and formyl. Where an alkyl group is substituted by an aryl group, this is sometimes referred to as an aralkyl group. Typically, aralkyl groups comprise a C₁₋₆ alkyl group substituted by an optionally substituted aryl group.

Alternatively or additionally to the substituents referred to immediately above, substituents of alkyl, alkenyl or alkynyl (and alkylene, alkenylene and alkynylene) groups may in some embodiments confer notable advantageous water-solubilising properties upon the compounds of formula (I). Appropriate solubilising substituents (one more of which, typically one of which, may be present only substituted group) may, for example, be selected from the group comprising sulfonate, quaternary ammonium, sulfate, phosphonate, phosphate and carboxyl. Alternatively, solubilising groups may be carbohydrate residues, for example, monosaccharides. Where a water-solubilising substituent is present, this may be present as a substituent of an alkyl group, typically a C₁₋₆ alkyl group, constituting R₁, R₂, R₃ or R₄. Examples of water-solubilising substituted alkyl groups thus include C₁-C₆ alkyl carboxylates and sulfonates, such as —(CH₂)₂₋₄—SO₃⁻ and (CH₂)₂₋₄—CO₂⁻, for example —(CH₂)₂—CO₂, although it is notable that such solubilising substituents may be directly attached to the tricyclic core of compounds of formula (I) (as possibilities for substituents R₃ and R₄). Water solubility may be particularly advantageous when compounds of formula (I) are conjugated with, i.e. used to label, proteins or peptides.

By aryl is meant herein a radical formed formally by abstraction of a hydrogen atom from an aromatic compound. Aryl groups are typically monocyclic groups, unless the context specifically dictates to the contrary, for example phenyl, although bicyclic aryl groups, such as naphthyl, and tricyclic aryl groups, such as phenanthrene and anthracene, are also embraced by the term aryl. As known to those skilled in the art, heretoaromatic moieties are a subset of aromatic moieties that comprise one or more heteroatoms, typically O, N or S, in place of one or more carbon atoms and optionally any hydrogen atoms attached thereto. Consequentially, it will be understood that heteroaryl groups are a subset of aryl groups. Illustrative heteroaromatic moieties include pyridine, furan, pyrrole and pyrimidine. Further examples of heteroaromatic rings include pyridazine (in which two nitrogen atoms are adjacent in an aromatic 6-membered ring); pyrazine (in which two nitrogens are 1,4-disposed in a 6-membered aromatic ring); pyrimidine (in which two nitrogen atoms are 1,3-disposed in a 6-membered aromatic ring); or 1,3,5-triazine (in which three nitrogen atoms are 1,3,5-disposed in a 6-membered aromatic ring).

Aryl groups may be substituted one or more times with substituents selected from, for example, the group consisting of hydroxy, amino, halo, alkyl, aryl, (including heteroaryl), nitro, alkoxy, alkylthio, cyano, sulfhydryl, acyl and formyl.

By amido is meant herein either of the functional groups —NHCOR, or —CONHR, wherein R is hydrogen or an optionally substituted alkyl group.

By acyl is meant the functional group of formula —C(O)R, wherein R is an optionally substituted alkyl group.

By ester is meant a functional group comprising the moiety —OC(═O)—.

Alkoxy (synonymous with alkyloxy) and alkylthio moieties are of the formulae —OR and —SR respectively, wherein R is an optionally substituted alkyl group.

By carboxy is meant herein the functional group —CO₂H, which may be in deprotonated form (CO₂⁻).

By sulfonate is meant herein the functional group —SO₃⁻ (which is the deprotonated form of sulfonic acids (—SO₃H)), which may be in protonated form.

By formyl is meant a group of formula —CHO.

Halo is fluoro, bromo, chloro or iodo.

By amino group is meant herein a group of the formula —NH₂. Where one or both of the hydrogen atoms of an amino group is substituted with an alkyl group this provides a mono- or dialkyl-substituted amino group. One example of dialkyl-substituted amino group is wherein the two alkyl groups join to form an alkylene diradical, derived formally from an alkane from which two hydrogen atoms have been abstracted, typically from terminal carbon atoms, whereby to form a ring together with the nitrogen atom of the amine. As is known, the diradical in cyclic amines need not necessarily be alkylene: morpholine (in which the alkylene is —(CH₂)₂O(CH₂)₂—) is one such example from which a cyclic amino substituent may be prepared.

References to amino and mono- or dialkyl-substituted amino groups herein are also to be understood as embracing within their ambit protonated derivatives of the amines resultant from compounds comprising such amino groups. Examples of the latter may be understood to be salts such as hydrochloride salts.

A quaternary ammonium group is a substituent comprising a nitrogen atom and three optionally substituted alkyl groups, with the resultant four bonds to the nitrogen atom conferring permanent positive charge.

One or more linker groups may be attached to the tricyclic core of the compounds of formula (I). These may be either unsubstituted (when L is hydrogen) or substituted with a target bonding group K. Typically, linking groups J comprise unbranched chains of atoms connecting group L with the tricyclic core of the compounds of formula (I). Each linking group J typically comprises from 1 to 40 (for example from 1 to 10) chain atoms comprising carbon, and optionally nitrogen, oxygen, sulfur and/or phosphorus. For example, the chain may be a substituted or unsubstituted (typically unsubstituted) alkylene (e.g. methylene, ethylene or propylene), alkenylene (e.g. ethenylene or propenylene), alkyleneoxy chain (e.g. —O(CH₂)₄—), or an alkyleneanecarboxamido chain, such as acetamido. Where group R₁ comprises a linker group J, the atom of linker group J attached to the tricyclic core of the compounds of formula (I) is generally a carbon atom.

The target bonding group K is a reactive or functional group, which allows the compound of formula (I) to be reacted under suitable conditions with a target molecule, e.g. a biological molecule. A reactive group of a compound according to formula (I) can react under suitable conditions with a functional group of, for example, a biological molecule; a functional group of a compound according to formula (I) can react under suitable conditions with a reactive group of, for example, a biological molecule. It is possible, according to either of these conjugation strategies, to label a target compound, e.g. a desired biological molecule, with a compound of formula (I).

Where K is a reactive group, this may be selected from succinimidyl ester, sulpho-succinimidyl ester, isothiocyanate, maleimide, haloacetamide, acid halide, vinylsulfone, dichlorotriazine, carbodimide, hydrazide, phosphoramidite pentafluoro phenyl ester and alkyl halide. Where K is a functional group, this may be selected from hydroxy, amino, sulfhydryl, imidazole, carboxyl, carbonyl (including aldehyde, ketone and thioester), phosphate, thiophosphate and aminooxy. It will be understood that K may become modified when conjugating to a biological molecule, for example an amino group may become part of an amide group, or a carboxyl may become part of an ester group. By virtue of these reactive and functional groups the compounds of formula (I) may be reacted with, and covalently bound to, biological molecules. The skilled addressee readily knows which functional/reactive groups are capable of reacting with corresponding reactive/functional groups of the biological molecule to which the compound of formula (I) is to be coupled/conjugated.

If a group R₂ is present, a counter ion X will also be present. There is no particular limitation to the nature of counter ions X; these may be any convenient counterion. For example, X may be a halide, in particular, chloride, bromide or iodide, tosylate, methyl sulfonate or an alkyl carboxylate, for example acetate or trifluoroacetate. Other examples will be evident to the skilled person. According to particular embodiments of the invention, R₂ and counter ion X are present.

Particular compounds of formula (I) in accordance with embodiments of all aspects of the invention are as defined above as the fourth aspect of the invention, according to which compounds of formula (I) comprise R¹=hydrogen or J-H; R²=J-L; and R³=hydrogen or J-K. According to other embodiments, compounds comprise a:

(i) group R₂ which is either an alkyl group, for example a C₁₋₆ alkyl group such as methyl, ethyl or propyl, for example methyl, or is of formula -J-K, for example wherein J is an alkylene linker group comprising from 1 to 6 carbon atoms, for example methylene, ethylene, propylene or butylene, and which according to particular embodiments as ethylene; and/or wherein the target bonding group is carboxyl; and/or

(ii) group R₁ which is of formula -J-K, for example wherein J is an alkylene linker group comprising from 1 to 6 carbon atoms, for example methylene, ethylene, propylene or butylene, and which according to particular embodiments as ethylene; and/or the target bonding group is carboxyl.

More particular embodiments of the foregoing embodiments of the compounds of the fourth aspect of the invention comprise a group R₂ which is either an alkyl group, for example a C₁₋₆ alkyl group such as methyl, ethyl or propyl, for example methyl, or is of formula -J-K, for example wherein J is an alkylene linker group comprising from 1 to 6 carbon atoms, for example methylene, ethylene, propylene or butylene, and which according to particular embodiments as ethylene; and optionally the target bonding group is carboxyl.

The particular and more particular embodiments of the compounds immediately hereinbefore defined according to (i) and (ii) may further comprise (iii) one R³ or R⁴ is a group of formula -J-K, for example wherein J is an alkylene linker group comprising from 1 to 6 carbon atoms, for example methylene, ethylene, propylene or butylene, and which according to particular embodiments as ethylene; and/or the target bonding group is carboxyl.

According to particular embodiments of (i) to (iii) described immediately hereinbefore, only one target bonding group is present, for example carboxyl. Typically, the target bonding group in these and other embodiments of the invention is connected to the remainder of the compound via a group J.

According to particular embodiments of all aspects of the invention, the compound of formula (I) is a dye having one of the formulae (Ill), (IV), (V), (VI) or (VII) shown below, wherein X⁻ is as described hereinbefore.

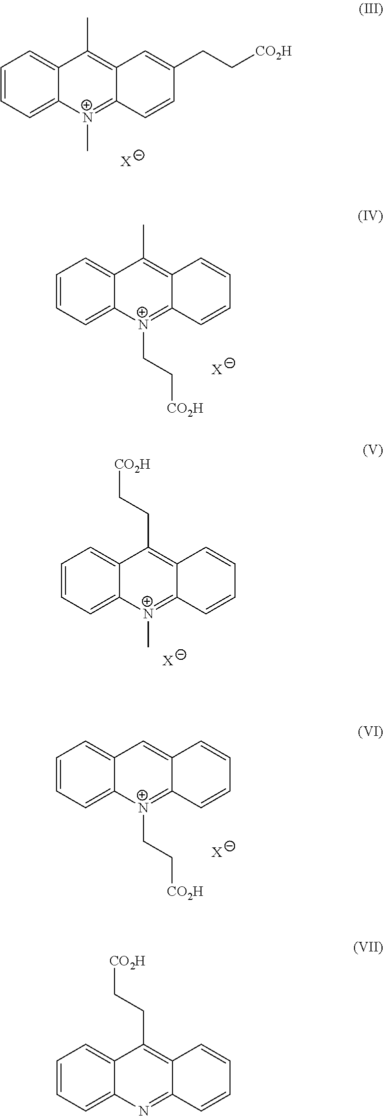

It will be appreciated that some of the fluorescent dyes described herein may contain a charge, for example, at a quaternary amino group, which this may be used to form salts or to bind negatively charged molecules such as DNA and/or RNA.

The compounds of formula (I) described herein may be readily synthesised by those of normal skill. The target bonding group may be introduced into the compound of formula (I) at the beginning of the synthesis of the compound (for example prior to the construction of the tricyclic core), or introduced to the tricyclic or after its construction. For example, where substituent R² comprises a target bonding group, this may be introduced by reaction of a precursor to the compound of formula (I) absent an R² group, by quaternisation of the central nitrogen of the tricycle with an appropriate precursor to R² (and X⁻). Representative syntheses, which the skilled person will be able to adapt readily to make other compounds of formula (I) as appropriate, are described below.

Suitable biological molecules with which the compounds of formula (I) may be conjugated, for example to provide conjugates useful according to the first, second, third and sixth aspects of the invention, and of the fifth aspect, include, but are not limited to the group consisting of antibodies, lipids, proteins, peptides, carbohydrates, nucleotides and oxy or deoxy polynucleic acids which contain or are derivatised to contain one or more of an amino, sulphydryl, carbonyl (including aldehyde and ketone), hydroxyl, carboxyl, phosphate, thiophosphate, aminoxy and hydrazide groups, microbial materials, drugs, hormones, cells, cell membranes and toxins.

Particularly preferred biological molecules for labelling with the fluorescent dyes of formula (I) described herein are peptides or proteins. The skilled addressee is aware of methods which allow labelling at a specific site in a synthesised peptide (see e.g. Bioconjugate Techniques, G. T. Hermanson, Academic Press (1996)). The conjugates described herein may comprise a cell entry peptide. The cell entry peptide may be Penetratin (Cyclacel, UK), for example TAT or Chariot.

The dyes of formula (I) are particularly suitable for use as fluorescence lifetime dyes. In accordance with the present invention, the term lifetime dye is intended to mean a dye having a measurable fluorescence lifetime, defined as the average amount of time that the dye remains in its excited state following excitation (Lackowicz, J. R., Principles of Fluorescence Spectroscopy, Kluwer Academic/Plenum Publishers, New York, (1999)).

With regard to the first aspect of the invention, the dyes of formula (I) described herein are of particular use in many biochemical and/or cell-based assays in which fluorescence lifetime may be measured, whereby to allow detection of target material, e.g. of a biological molecule, with which the dye of formula (I) may be conjugated. Consequentially, an alternative description of the first aspect of the invention may be considered to be a method comprising measuring the fluorescence lifetime of a conjugate of a compound of formula (I) and a target molecule, which target molecule may be a biological molecule as described herein. For example, uses and methods according to the first aspect of the invention may involve assays such as those described in WO 02/099424 A2 and WO 03/089665 A1, or methods of the second or third aspects of the present invention, including those embodiments described below.

In an assay in accordance with the second aspect of the invention, for example, a fluorescent dye of formula (I) can be used to detect whether or not an analyte is present in a sample. This is achieved by contacting a conjugate of (i) a known binding partner of the analyte and (ii) a compound of formula (I) as defined herein, for example but not necessarily in accordance with the fourth aspect of the invention, with a sample that may or may not comprise the analyte. The fluorescence intensity/fluorescence lifetime arising from the presence of the fluorescent dye of formula (I) within the conjugate is measured before and after contact with the sample. Any modulation of the measured fluorescence intensity or fluorescence lifetime may be correlated with the presence of the analyte and thus used to assay for it.

According to some embodiments, the conjugate used according to the second aspect of the invention may comprise a specific binding partner of the analyte the presence of which it is desired to be able to detect. Illustrative pairs of analyte-specific binding partners include protein/protein, protein/nucleic acid, nucleic acid/nucleic acid, protein/small molecule and nucleic acid/small molecule partners; or antibodies/antigens, lectins/glycoproteins, biotin/streptavidin, hormone/receptor, enzyme/substrate or co-factor, DNA/DNA, DNA/RNA and DNA/binding protein. This list is not exhaustive and other combinations will be evident to the skilled person. It will be understood that, for each combination, either member of the combination may be the analyte or the known binding partner of the analyte. For example, according to embodiments of the second aspect of the invention, therefore, the analyte may be an enzyme and the known binding partner a substrate or cofactor therefor; or the analyte may be a substrate or cofactor for an enzyme, which enzyme is the known binding partner.

In an assay in accordance with the third aspect of the invention, for example, a fluorescent dye of formula (I) can be used to measure the activity of an enzyme in the presence of a conjugate comprising a compound of interest and a dye of formula (I). This is achieved by contacting such a conjugate with the enzyme. The fluorescence intensity/fluorescence lifetime arising from the presence of the fluorescent dye of formula (I) within the conjugate is measured before and after contact with the enzyme. Any modulation of the measured fluorescence intensity or fluorescence lifetime may be correlated with the activity of the enzyme.

According to some embodiments, the compound of the conjugate used according to the third aspect of the invention may be a biological molecule, e.g. a substrate or cofactor for the enzyme, for example a substrate for the enzyme. For instance, the biological molecule may be susceptible to phosphorylation and the enzyme a kinase. The substrate may be a peptidic substrate, for example one comprising between 4 and 20 amino acid residues. Such peptidic substrates may also constitute the analyte or known binding partner in accordance with the second aspect of the invention.

Such substrates or cofactors may comprise a moiety that modulates (typically but not necessarily reduces) the fluorescence and/or fluorescence lifetime of the compound of formula (I) whilst the conjugate remains intact. Upon separation of the fluorescence (lifetime)-modulating moiety from the compound of formula (I), for example upon cleavage of the conjugate by the enzyme, fluorescence intensity and/or fluorescence lifetime may then typically increase, with such an increase being used to measure the activity of the enzyme.

For example, aromatic amino acids such as tryptophan may be used to modulate the fluorescence intensity and/or fluorescence lifetime of a dye of formula (I). A conjugate of a dye of formula (I) and a peptidic substrate for a peptidase enzyme comprising a tryptophan residue, which residue is cleaved from the peptidic substrate upon action of the peptidase enzyme, may be used to measure the activity of an enzyme. Additional methods for quenching the fluorescence of fluorescently labelled peptides have been disclosed. Thus WO 02/081509, for example, describes the use of tryptophan, tyrosine or histidine residues to internally quench fluorescence intensity within fluorescently labelled peptides. Phenylalanine may also be used for this purpose, as may be naphthylalanine, an unnatural amino acid variant thereof, or rather unnatural aromatic amino acids. The peptides can be used to detect endo- and exo-peptidase activity. Additional methods relating to fluorescent lifetime measurements are described in WO 03/089663 A2, to which the skilled reader is directed. The techniques described therein can be applied to the methods disclosed herein.

Alternatively, the conjugate may be of a substrate for a kinase and a fluorescent dye of formula (I), i.e. which conjugate may be phosphorylated by the kinase. Such conjugates may comprise fluorescence-modulating moieties as described herein, for example aromatic amino acids (as described immediately above). Where the fluorescence-modulating moiety is tyrosine, for instance, phosphorylation of its phenolic hydroxyl group serves to convert it to a phosphate moiety, whereby to modulate the fluorescence-modulating effect of the tyrosine's aromatic ring so as to effect an increase in the fluorescence of the substrate.

As a variant of the embodiments of the invention described immediately above, the conjugate may comprise a substrate susceptible to phosphorylation by a kinase, which phosphorylation serves to permit introduction of a fluorescence-modulating moiety. For example, phosphorylation of side chain of an amino acid residue that need not be fluorescence-modulating (e.g. serine or threonine) may be used in tandem with exposure to fluorescence-modulating moieties constituted by polydentate ligands coordinated to an iron (III) ions, as is described in WO 2009/001051 A2. For example, the polydentate ligand may be aromatic- or heteroaromatic-containing and/or bi- or tridentate. In this way the contacting in accordance with the third aspect of the invention may take place in the presence of (in addition to the conjugate) chelates formed between iron (III) ions and such ligands. According to particular embodiments, the chelates are of phenylmalonic acid or 2-hydroxyacetophenone. Without wishing to be bound by theory, it is understood that iron (III) ions bind according to these embodiments of the invention to the phosphate moiety through electrostatic interactions, bringing the aromatic ligands into proximity with the fluorophore whereby to effect fluorescence modulation and thus a decrease in the fluorescence of the substrate.

According to alternative embodiments of the third aspect of the invention, the enzyme may be used to effect ligation between the conjugate to a compound that modulates (typically but not necessarily reduces) the fluorescence and/or fluorescence lifetime of the compound of formula (I) upon ligation. Upon reaction of the enzyme, therefore, fluorescence intensity and/or fluorescence lifetime may then typically decrease, with such a decrease been used to measure the activity of the enzyme. For example, the modulating compound may be a peptide comprising a tryptophan, tyrosine, histidine, naphthylalanine or phenylalanine residue such that ligation serves to bring the residue into proximity with the dye of formula (I), whereby to produce the fluorescence intensity and/or fluorescence lifetime of the resultant ligation product. Other fluorescence-modulating moieties include, for example, naphthyl, indolyl and phenoxy groups.

According to embodiments of the second aspect of the invention (when either the analyte or known binding partner is an enzyme) and third aspect of the invention, the enzyme may be selected from the group consisting of kinases, phosphatases proteases, esterase, peptidases, amidases, nucleases and glycosidases, for example kinases and phosphatases. For example, the enzyme may be selected from the group consisting of angiotensin converting enzyme (ACE), caspase, cathepsin D, chymotrypsin, pepsin, subtilisin, proteinase K, elastase, neprilysin, thermolysin, asp-n, matrix metallo protein 1 to 20, papain, plasmin, trypsin, enterokinase and urokinase.

According to particular embodiments of the third aspect of the invention, the method may be used to determine the effect, if any, a test compound has upon the activity of the enzyme. According to these embodiments, a method of the third aspect of the invention is carried out both in the presence and in the absence of the test compound. Any difference in activity of the enzyme found is indicative of an effect upon the activity of the enzyme exhibited by the compound. For example, the compound may act as an inhibitor or as a promoter of the enzyme. According to particular embodiments, a plurality of methods according to the third aspect of the invention may be conducted with different quantities or concentrations of the test compound. In this way, for example, the IC₅₀ value may be determined, where the test compound is an inhibitor of the enzyme.

For cell-based assays, the assays may be carried out on live cells or using cell components, such as cell wall fragments. Any cell may be utilised including prokaryotic and eukaryotic cells, especially mammalian and human cells.

Methods and uses of the invention may be carried out in liquid media, generally solutions, of any convenient pH, which will typically be in the range of approximately 5 to 9. The liquid media are typically aqueous. For example, water or appropriate acidic or alkaline solutions, for example buffered solutions such as phosphate-buffered saline (PBS) solutions may be used.

According to particular embodiments of the present invention, different dyes of formula (I), for example conjugated to different compounds, may be used simultaneously according to the various aspects of the present invention. Where the fluorescence intensity and/or fluorescence lifetime of the dyes allow those to be distinguished from one another, this permits multiplexing. Further details may be found in WO 03/089663 A2 (infra).

The methods of the present invention may typically be performed in the wells of a multiwell plate, e.g. a microtitre plate having 24, 96, 384 or higher densities of wells e.g. 864 or 1536 wells. A suitable instrument is the Edinburgh Instruments Nanotaurus Fluorescence Lifetime Platereader. Alternatively, the methods may be conducted in assay tubes or in the microchannels of a multifluidic device.

Conventional detection methods can be employed to measure fluorescence intensity and/or the lifetime of the label. These methods include instruments using photo-multiplier tubes as detection devices. Several approaches are possible using these methods; e.g.

i) methods based upon time correlated single photon counting (cf. Principles of Fluorescence Spectroscopy, (Chapter 4) ed. J R Lakowicz, Second Edition, 1999, Kluwer/Academic Press);

ii) methods based upon frequency domain/phase modulation (cf. Principles of Fluorescence Spectroscopy, (Chapter 5) ed. J R Lakowicz, Second Edition, 1999, Kluwer/Academic Press); and

iii) methods based upon time gating (cf. Sanders et al., (1995) Analytical Biochemistry, 227 (2), 302-308).

A suitable device is the Edinburgh Instruments FLS920 fluorimeter, Edinburgh Instruments, UK.

Measurement of fluorescent intensity may be performed by means of a charge coupled device (CCD) imager, such as a scanning imager or an area imager, to image all of the wells of a multiwell plate. The LEADseeker™ system features a CCD camera allowing imaging of high density microtitre plates in a single pass. Imaging is quantitative and rapid, and instrumentation suitable for imaging applications can now simultaneously image the whole of a multiwell plate.

All publications (patent and non-patent) referred to herein are incorporated by reference in their entireties, as if the entire contents of each reference was set forth herein in its entirety.

The invention is now illustrated by the following non-limiting examples.

### Example 1

Fluorescence lifetimes for a variety of acridine derivatives studied are reported in Table 1. Fluorescence lifetimes were determined by time-correlated single photon counting (TCSPC) acquisition using an Edinburgh Instruments Nanotaurus fluorescence lifetime platereader, using excitation laser 405 nm and either a 438 nm band pass, 450 nm band pass or 473 nm long pass emission filter for detection.

TABLE 1
Long lifetime fluorescent dyes

| | | Fluorescence Lifetime (ns) | | |
|---|---|---|---|---|
| | | 1 μM in | 1μM in | 1 μM in 50 mM |
| Entry | Structure | PBS | water | AcOH solution |
| 1 | 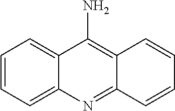 [SMILES: Nc1c2ccccc2nc2ccccc12] | 15.6 | 15.7 | 15.6 |
| 2 | 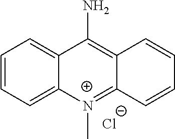 [SMILES: C[n+]1c2ccccc2c(N)c2ccccc21 ; [Cl-]] | 16.3 | 16.4 | 16.4 |
| 3 | 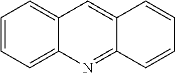 [SMILES: c1ccc2nc3ccccc3cc2c1] | 18.8 | 10.5 | 29.4 |
| 4 | 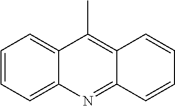 | 11.7 | 29.1 | 29.1 |
| 5 | 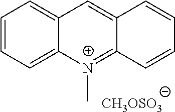 | 31.9 | 14.1 | 32.1 |
| 6 | 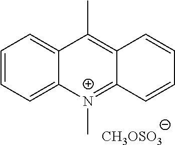 | 26.2 | 30.0 | 29.8 |

### Example 2

### Synthesis of 9,10-dimethylacridin-10-ium methyl sulfate

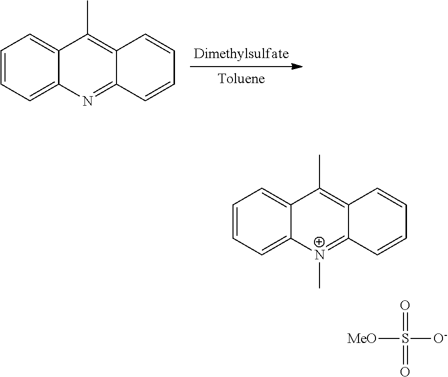 [SMILES: Cc1c2ccccc2nc2ccccc12 ; Cc1c2ccccc2[n+](C)c2ccccc12 ; COS(=O)(=O)[O-]]

9-Methylacridine (440 mg, 2.3 mmol) was dissolved in toluene (10 ml) and dimethyl sulfate (652 μl, 6.9 mmol) was then added. The mixture was heated at reflux for 2 hours and then allowed to cool to room temperature. The yellow precipitate was isolated by filtration and washed with diethyl ether, followed by diethyl ether/dichloromethane to afford the product as a yellow-green powder (720 mg, quant.). Analyses by ¹H NMR and MS conformed to structure.

### Example 3

### Fluorescence Analysis of 9,10-dimethylacridin-10-ium methyl sulfate

Steady state measurements were performed on an Edinburgh Instruments FLS920 steady state fluorimeter, with excitation wavelength of 350 nm and emission wavelength of 490 nm. Fluorescence lifetimes were determined by time-correlated single photon counting (TCSPC) acquisition using an Edinburgh Instruments Nanotaurus fluorescence lifetime platereader, using excitation laser 405 nm and a 473 nm long pass emission filter for detection.

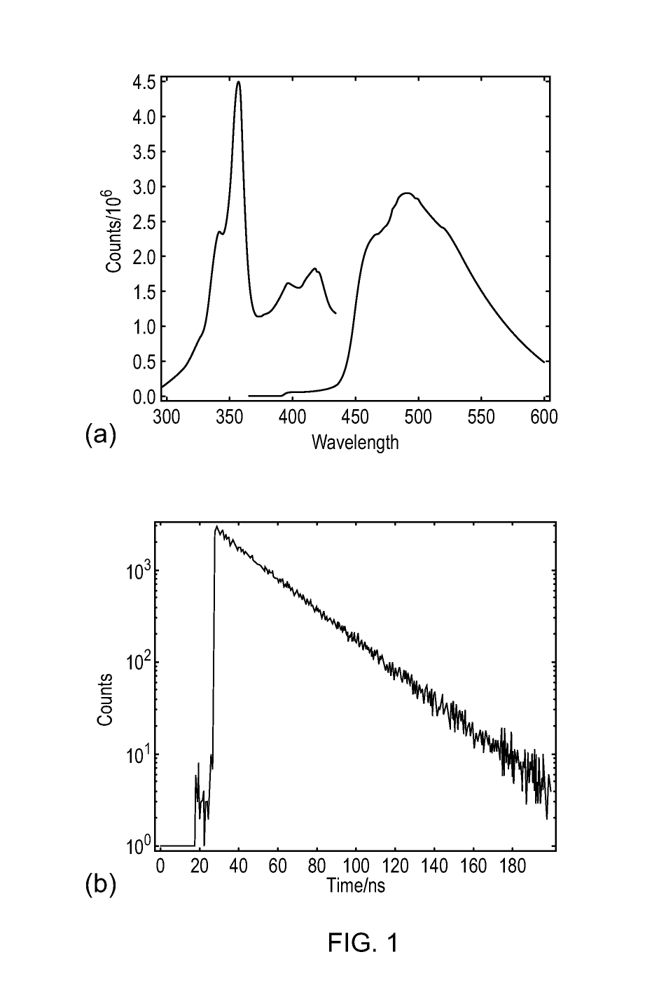

The fluorescence excitation and emission spectra were measured at 1 μM 9,10-dimethylacridin-10-ium methyl sulfate concentration in phosphate buffered saline (PBS) solution pH 7.4. The fluorescence excitation and emission spectra, and fluorescence lifetime decay curve are shown in FIG. 1(a) and FIG. 1(b) respectively. 9,10-Dimethylacridin-10-ium methyl sulfate has an advantageously long fluorescence lifetime of circa 25-30 ns. In addition, the magnitude of the fluorescence lifetime was maintained across the three buffer systems tested (see Table 1, entry 6).

### Example 4

### pH Stability Studies

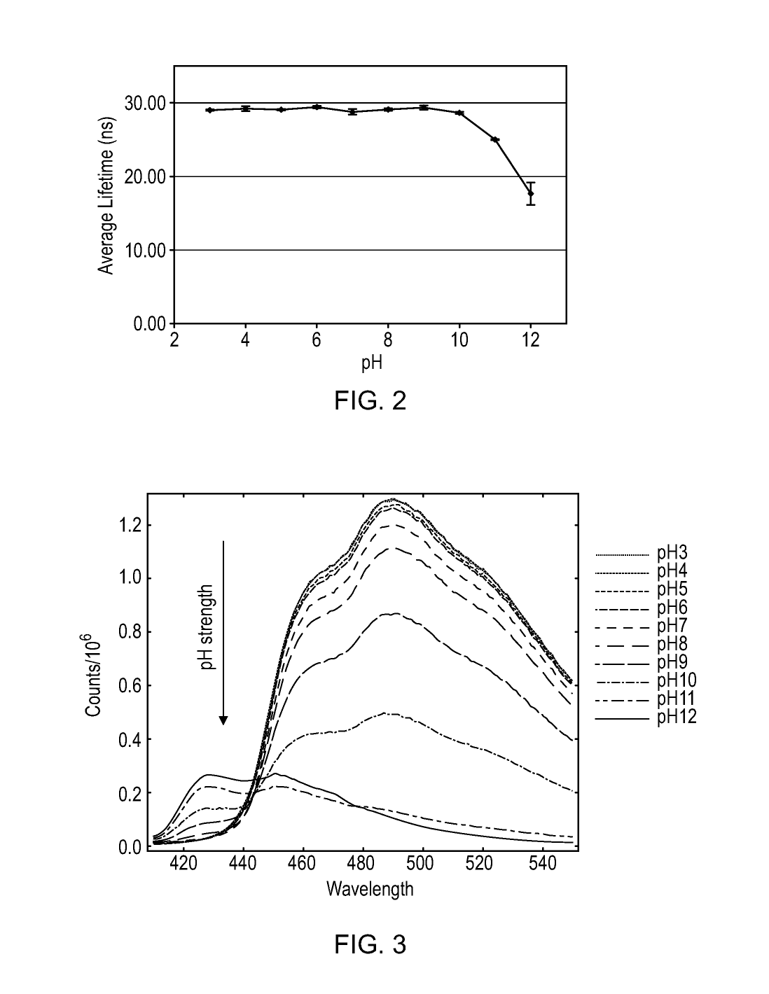

An important criteria of fluorescence dyes suitable for use in biochemical and cell-based assays is the advantageousness of the fluorescence lifetime being independent of pH in the physiological range 5 to 9. The fluorescence lifetime of the 9,10-dimethylacridin-10-ium methyl sulfate was stable across the pH range 3 to 10 (see FIG. 2).

The fluorescence emission profile was measured as a function of pH upon excitation at 405 nm (FIG. 3). An emission maximum of 460 nm and 490 nm was observed for all solutions at pH 3 to 10, but for solutions >pH 10 the emission maximum shifted to 425 and 450 nm. This change in fluorescence emission profile and fluorescence lifetime for pH >10 may represent deprotonation of the acidic methyl protons at the 9-position under basic conditions, furnishing 9-methyleneacridine as shown below in Scheme 1: These methyl protons are reported to be unusually acidic with a pKa akin to acetic acid (Tanaka, Y. et al., J. Org. Chem., 2001, 66, 2227).

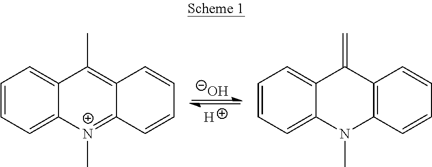

### Example 5

### Derivatisation of 9,10-dimethylacridin-10-ium methyl sulfate with a carboxylic acid moiety

To facilitate the attachment of fluorophores to biological molecules such as peptides and proteins, carboxylic acid derivatives of the fluorophore are often desirable: such compounds will react with an amino functionality on peptides and proteins to allow the conjugation through amide bond formation. A series of acridine and acridinium compounds were designed, all of which incorporate a carboxylic acid moiety to enable attachments to a peptide via an amide bond linkage (see Table 2).

TABLE 2
Carboxylic acid derivatised acridine/acridinium dyes

| Target | Structure | Name |
|---|---|---|
| 1 (IIII) | 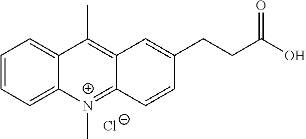 [SMILES: Cc1c2ccccc2[n+](C)c2ccc(CCC(=O)O)cc12 ; [Cl-]] | 2-(2-carboxyethyl)-9,10- dimethylacridin-10-ium chloride |
| 2 (VI) | 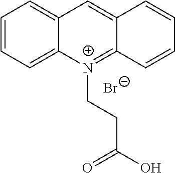 [SMILES: O=C(O)CC[n+]1c2ccccc2cc2ccccc21 ; [Br-]] | 10-(2-carboxyethyl)acridin-10-ium bromide |
| 3 (IV) | 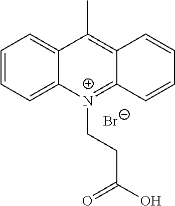 [SMILES: Cc1c2ccccc2[n+](CCC(=O)O)c2ccccc12 ; [Br-]] | 10-(2-carboxyethyl)-9- methylacridin-10-ium bromide |
| 4 (VII) | 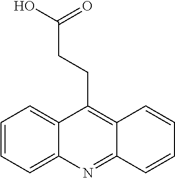 [SMILES: O=C(O)CCc1c2ccccc2nc2ccccc12] | 3-(acridin-9-yl)propionic acid |
| 5 (V) | 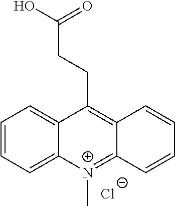 [SMILES: C[n+]1c2ccccc2c(CCC(=O)O)c2ccccc21 ; [Cl-]] | 9-(2-carboxyethyl)-10- methylacridin-10-ium chloride |

### Derivatisation of 9,10-dimethylacridin-10-ium methyl sulfate with a carboxylic acid at the 2-position

### Target 1: 2-(2-Carboxyethyl)-9,10-dimethylacridin-10-ium chloride

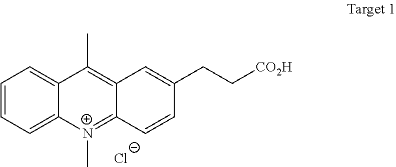 [SMILES: Cc1c2ccccc2[n+](C)c2ccc(CCC(=O)O)cc12 ; [Cl-]]

Target 1 was synthesised in 6 steps to afford 2-(2-carboxyethyl)-9,10-dimethylacridin-10-ium as the chloride salt (Scheme 2). The first three steps proceeded well to furnish the acridine ring framework with the desired propionic acid linker attached. Unfortunately however, the methyl ester group was hydrolysed during the final cyclisation step (step 3) and it was decided to convert the acid back the ester for ease of handling and to aid solubility for the subsequent steps. N-methylation was carried out using methyl iodide in a sealed tube and finally hydrolysis of the ester furnished the desired target.

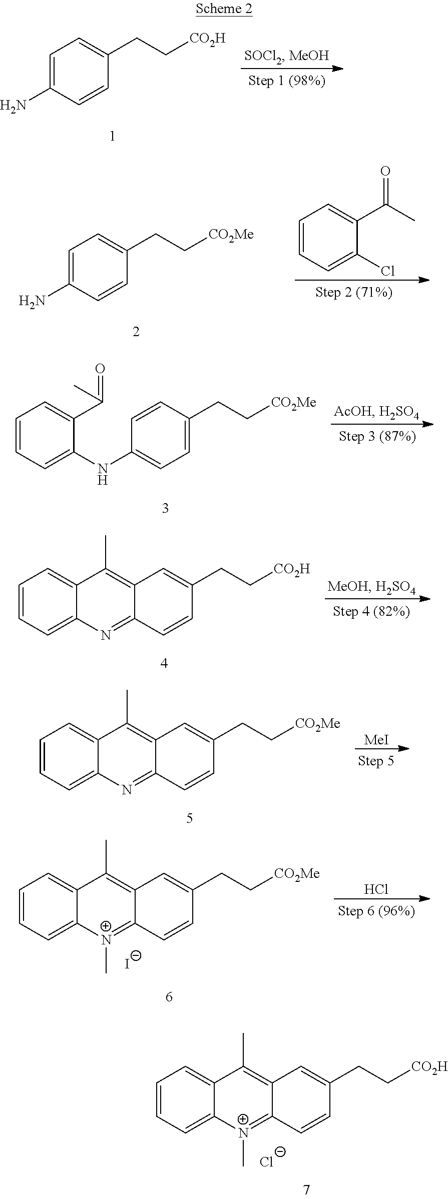

### Step 1:

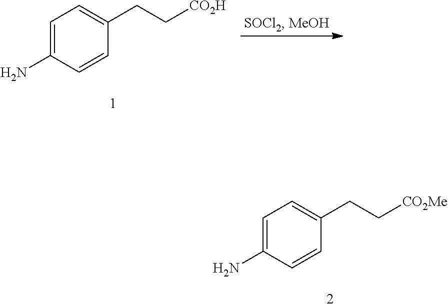 [SMILES: Nc1ccc(CCC(=O)O)cc1 ; COC(=O)CCc1ccc(N)cc1]

3-(4-Amino-phenyl)-propionic acid 1 (5 g, 30.3 mmol) was stirred at room temperature in SOCl₂ (20 mL) for 3 h. The SOCl₂ was removed in vacuo and the residue re-dissolved carefully in MeOH and stirred for 5 min. The MeOH was removed in vacuo and the residue dissolved in DCM, washed with a 10% K₂CO₃ solution, dried over MgSO₄ and concentrated in vacuo. Purification by column chromatography (heptane/EtOAc, 70:30) furnished 3-(4-amino-phenyl)-propionic acid methyl ester 2 as a colourless solid (5.37 g, 29.9 mmol, 98%). ¹H NMR (500 MHz, CDCl₃) δ 6.98 (d, J=8.4 Hz, 2H, ar), 6.62 (d, J=8.4 Hz, 2H, ar), 3.66 (s, 3H, OCH₃), 2.83 (t, J=7.8 Hz, 2H, CH₂), 2.57 (t, J=7.8 Hz, 2H, CH₂); ¹³C NMR (125 MHz, CDCl₃) δ 173.56 (C═O), 144.64 (C), 130.54 (C), 129.10 (CH×2), 115.30 (CH×2), 51.54 (00H₃), 36.15 (CH₂), 30.18 (CH₂); ESI m/z=180.17 (M+H)+, calc. for C₁₀H₁₃NO₂=179.09

### Step 2:

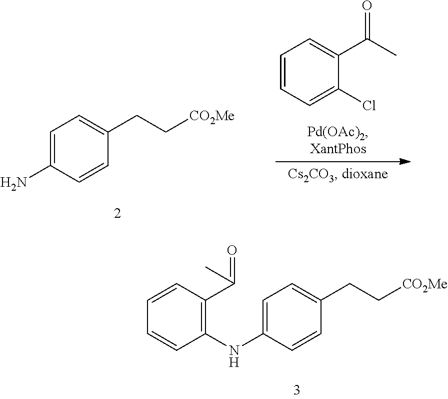

3-(4-Amino-phenyl)-propionic acid methyl ester 2 (5.37 g, 29.9 mmol), 2-chloroacetophenone (4.09 mL, 31.5 mmol), Pd(OAc)₂ (338 mg, 1.51 mmol, 5 mol %), XantPhos (864 mg, 1.49 mmol, 5 mol %) and Cs₂CO₃ (14.6 g, 44.8 mmol) were dissolved in dioxane (60 mL). The mixture was stirred at 110° C. for 20 h. After cooling, the mixture was filtered through celite, washed with DCM and the solvent removed in vacuo. Purification by column chromatography (heptane/EtOAc, 80:20) furnished 3-[4-(2-acetyl-phenylamino)-phenyl]-propionic acid methyl ester 3 as a yellow oil (6.26 g, 21.1 mmol, 71%). ¹H NMR (500 MHz, CDCl₃) δ 10.41 (br, NH), 7.73 (dd, J=8.1, 1.4 Hz, 1H, ar), 7.22 (dt, J=8.5, 1.4 Hz, 1H, ar), 7.11 (m, 5H, ar), 6.63 (dt, J=7.5, 1.1 Hz, 1H, ar), 3.61 (s, 3H, OCH₃), 2.87 (t, J=7.8 Hz, 2H, CH₂), 2.57 (t, J=7.8 Hz, 2H, CH₂), 2.55 (s, 3H, COCH₃); ¹³C NMR (125 MHz, CDCl₃) δ 201.16 (C), 173.34 (C), 148.24 (C), 138.49 (C), 136.31 (C), 134.55 (CH), 132.53 (CH), 129.21 (CH×2), 123.56 (CH×2), 118.83 (C), 116.29 (CH), 114.08 (CH), 51.64 (OCH₃), 35.75 (CH₂), 30.42 (CH₂), 28.11 (COCH₃); ESI m/z=280.25 (M+H)+, calc. for C₁₈H₁₉NO₃=279.35

### Step 3:

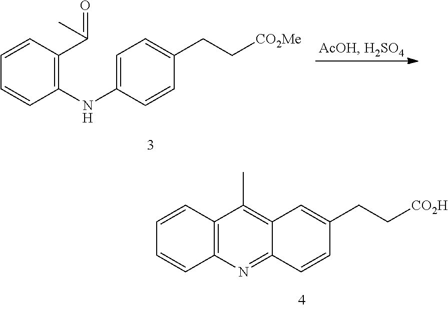 [SMILES: COC(=O)CCc1ccc(Nc2ccccc2C(C)=O)cc1 ; Cc1c2ccccc2nc2ccc(CCC(=O)O)cc12]

3-[4-(2-Acetyl-phenylamino)-phenyl]-propionic acid methyl ester 3 (2.20 g, 7.40 mmol) was dissolved in acetic acid (30 mL). H₂SO₄ (2 mL) was added and the mixture was stirred at reflux for 3 h. After cooling to room temperature, solid K₂CO₃ was added until pH 6 was obtained. The solid was isolated by filtration and dried under vacuum to give 3-(9-methyl-acridin-2-yl) propionic acid 4 as a light yellow solid (1.71 g, 6.45 mmol, 87%). This material was used without further purification.

### Step 4:

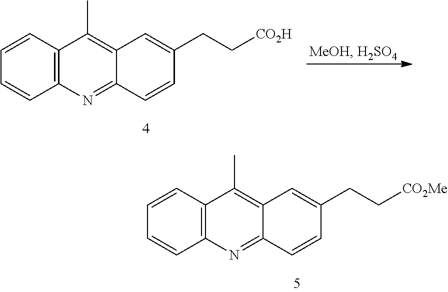 [SMILES: Cc1c2ccccc2nc2ccc(CCC(=O)O)cc12 ; COC(=O)CCc1ccc2nc3ccccc3c(C)c2c1]

3-(9-Methyl-acridin-2-yl) propionic acid 4 (1.35 g, 5.09 mmol) was dissolved in MeOH (50 mL) and H₂SO₄ (2 mL) was added. The mixture was stirred at reflux for 4 h and then was poured into water. The product was extracted with DCM, dried over MgSO₄ and concentrated in vacuo to give 3-(9-methyl-acridin-2-yl)-propionic acid methyl ester 5 as a light brown solid (1.16 g, 4.15 mmol, 82%). ¹H NMR (500 MHz, CDCl₃) δ 8.14 (m, 3H, ar), 7.96 (s, 1H, ar), 7.68 (m, 1H, ar), 7.57 (m, 1H, ar), 7.47 (m, 1H, ar), 3.62 (s, 3H, OCH₃), 3.14 (t, J=7.8 Hz, 2H, CH₂), 3.04 (s, 1H, CH₃), 2.72 (t, J=7.8 Hz, 2H, CH₂); ¹³C NMR (125 MHz, CDCl₃) δ 173.18 (C), 184.21 (C), 147.61 (C), 141.43 (C), 137.53 (C), 131.19 (CH), 130.50 (CH), 130.26 (CH), 129.48 (CH), 125.70 (C), 125.47 (CH), 124.48 (CH), 122.65 (CH), 51.73 (OCH₃), 35.39 (CH₂), 31.47 (CH₂), 13.60 (CH₃); ESI m/z=280.25 (M+H)+, calc. for C₁₈H₁₇NO₂=279.33

### Step 5:

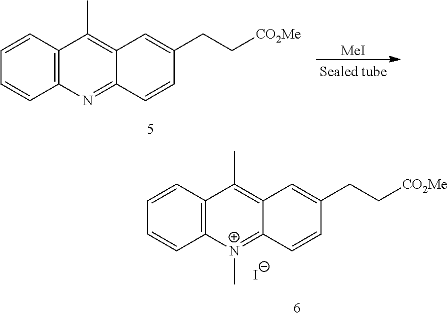 [SMILES: "MeI" ; COC(=O)CCc1ccc2nc3ccccc3c(C)c2c1 ; COC(=O)CCc1ccc2c(c1)c(C)c1ccccc1[n+]2C ; [I-]]

3-(9-Methyl-acridin-2-yl) propionic acid methyl ester 5 (120 mg, 0.43 mmol) was dissolved in Mel (3 mL). The mixture was stirred in a sealed tube at 90° C. for 20 h. The precipitate was collected by filtration and dried under vacuum to give a mixture of unreacted 5 and 2-(2-methoxycarbonyl-ethyl)-9,10-dimethyl-acridinium iodide 6 (15 /85). This mixture was purified by column chromatography (DCM/EtOH, 90:10). ¹H NMR (500 MHz, (CD₃)₂SO₂) δ 8.88 (d, J=8.6 Hz, 1H, ar), 8.72 (m, 3H, ar), 8.40 (dt, J =9.9, 7.5 Hz, 2H, ar), 8.01 (t, J=7.6 Hz, 1H, ar), 4.80 (s, 3H, NCH₃), 3.62 (s, 3H, OCH₃), 3.50 (s, 3H, CH₃), 3.25 (t, J=7.5 Hz, 2H, CH₂), 2.91 (t, J=7.5 Hz, 2H, CH₂); ¹³C NMR (125 MHz, (CD₃)₂SO₂) δ 172.46 (C), 159.65 (C), 140.24 (C), 139.93 (C), 139.65 (CH), 139.32 (C), 137.79 (CH), 128.05 (CH), 127.28 (CH), 126.07 (CH), 125.60 (C×2), 119.29 (CH), 119.20 (CH), 51.43 (OCH₃), 38.67 (CH₃), 34.13 (CH₂), 29.78 (CH₂), 16.40 (CH₃); ESI m/z=294.25 (M)+, calc. for C₁₃H₂₀NO₂=294.37

### Step 6:

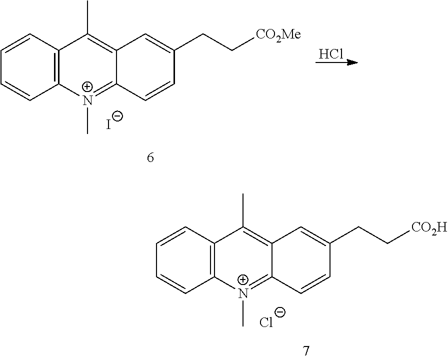 [SMILES: COC(=O)CCc1ccc2c(c1)c(C)c1ccccc1[n+]2C ; [I-] ; Cl ; Cc1c2ccccc2[n+](C)c2ccc(CCC(=O)O)cc12 ; [Cl-]]

2-(2-Methoxycarbonyl-ethyl)-9,10-dimethyl-acridinium iodide 6 (250 mg, 0.594 mmol) was dissolved in 6M HCl (10 mL) and the mixture was stirred at reflux for 3.5 h. Concentration in vacuo furnished 2-(2-carboxy-ethyl)-9,10-dimethyl-acridinium chloride 7 as a light brown solid (180 mg, 0.570 mmol, 96%). ¹H NMR (500 MHz, (CD₃)₂SO₂) δ 8.88 (d, J=8.7 Hz, 1H, ar), 8.72 (m, 3H, ar), 8.40 (m, 2H, ar), 8.01 (t, J=7.6 Hz, 1H, ar), 4.80 (s, 3H, NCH₃), 3.50 (s, 3H, CH₃), 3.22 (t, J=7.5 Hz, 2H, CH₂), 2.31 (t, J=7.5 Hz, 2H, CH₂); ¹³C NMR (125 MHz, (CD₃)₂SO₂) δ 173.52 (C), 159.56 (C), 140.63 (C), 139.88 (C), 139.74 (CH), 139.29 (C), 137.45 (CH), 128.04 (CH), 127.26 (CH), 125.97 (CH), 125.60 (C), 125.55(0), 119.24 (CH), 119.19 (CH), 38.68 (CH₃), 34.53 (CH₂), 29.91 (CH₂), 16.40 (CH₃); ESI m/z=280.26 (M)+, calc. for C₁₈H₁₈NO₂+=280.34

### Derivatisation at the 10-position

### Target 2: 10-(2-Carboxyethyl)acridin-10-ium bromide

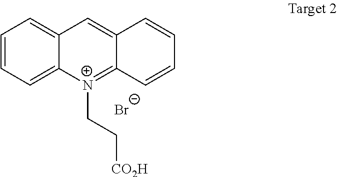 [SMILES: O=C(O)CC[n+]1c2ccccc2cc2ccccc21 ; [Br-]]

Target 2 was synthesised in 4 steps to afford 10-(2-carboxyethyl)acridin-10-ium bromide (Scheme 3). The propiolactone 8 was converted to 3-trifluoromethanesulfonyloxy-propionic acid benzyl ester 10 as described in Omura, S. et al., J. Antibiotics, 1992, 45, 1139. Subsequent reaction with acridine was performed following the protocol described in Fukuzumi, S. et al., J. Mater. Chem., 2005, 15, 372.

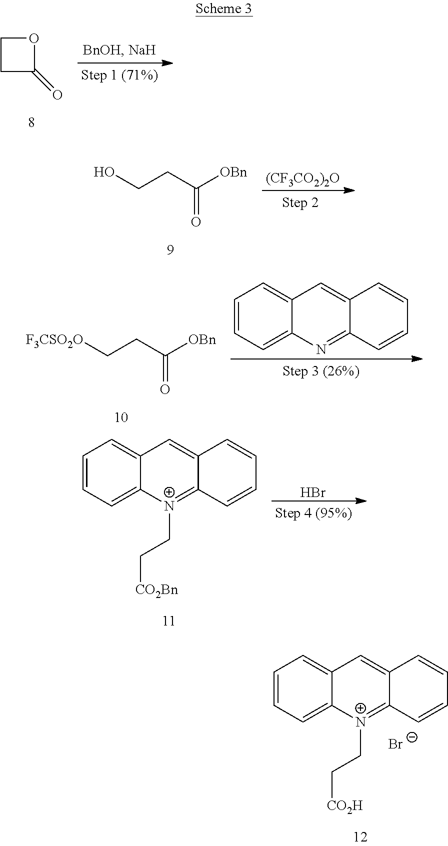

### Step 1

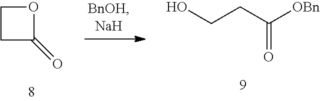 [SMILES: O=C1CCO1 ; O=C(CCO)OCc1ccccc1]

Benzyl alcohol (5 mL, 48.3 mmol) was cooled in an ice bath and 60% NaH (50 mg, 1.25 mmol) was added portionwise, the mixture was stirred at 0° C. for 30 min. Propiolactone 8 (0.5 mL, 7.97 mmol) was then added dropwise and the mixture was stirred at 0° C. for a further 30 min. The reaction was quenched by addition of 2M HCl (2 mL). The mixture was extracted with DCM, washed with water, dried over MgSO₄ and evaporated. The residue was purified by column chromatography (heptane/EtOAc, 80:20) to afford 3-hydroxy-propionic acid benzyl ester 9 as a colourless oil (1.02 g, 5.66 mmol, 71%). Analyses by ¹H NMR, ¹³C NMR and MS conformed to structure.

### Step 2

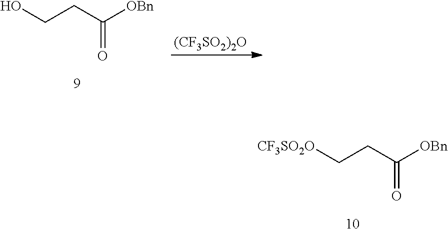

3-Hydroxy-propionic acid benzyl ester 9 (360 mg, 2.0 mmol) and pyridine (178 μL, 2.2 mmol) were dissolved in DCM (8 mL). The solution was cooled to −20° C. and triflic anhydride (368 μL, 2.2 mmol) was added dropwise. The mixture was stirred at −20° C. for 30 min and then at room temperature for 1 h. Concentration in vacuo and purification by rapid column chromatography (heptane/EtOAc, 80:20) afforded 3-trifluoromethanesulfonyloxy-propionic acid benzyl ester 10, which was used directly in the next step (estimated 2 mmol).

### Step 3

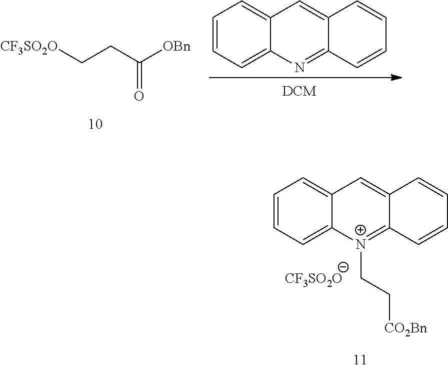

3-Trifluoromethanesulfonyloxy-propionic acid benzyl ester 10 (estimated 2 mmol) and acridine (360 mg, 2 mmol) were dissolved in DCM (10 mL) and the mixture was stirred at room temperature for 24 h. Concentration in vacuo afforded a black oil that was precipitated in heptane and isolated by filtration. Purification by column chromatography (DCM/EtOH, 95:5) afforded 10-(3-(benzyloxy)-3-oxopropyl)acridin-10-ium trifluoromethanesulphonate 11 as a yellow solid (260 mg, 0.529 mmol, 26%). ¹H NMR (500 MHz, CDCl₃) δ 9.99 (s, 1H, ar), 8.54 (dd, 4H, ar), 8.29 (t, 2H, ar), 7.83 (t, 2H, ar), 7.23 (m, 5H, ar), 5.70 (t, 2H, CH₂), 5.05 (s, 2H, CH₂), 3.25 (t, 3H, CH₂).

### Step 4

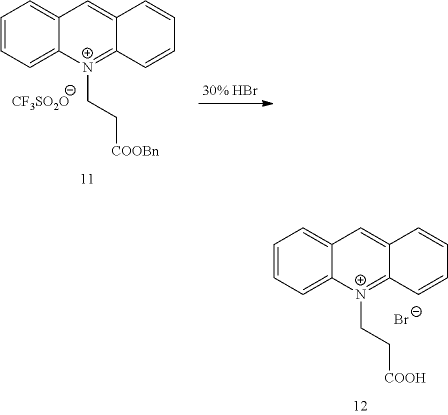

10-(3-(Benzyloxy)-3-oxopropyl)acridin-10-ium trifluoromethanesulphonate 11 (250 mg, 0.508 mmol) was dissolved in 30% HBr in acetic acid and the mixture was stirred at 50° C. for 2 h. Concentration in vacuo afforded 10-(2-carboxyethyl)acridin-10-ium bromide 13 as a light brown solid (160 mg, 0.482 mmol, 95%). ¹H NMR (500 MHz, (CD₃)₂SO₂) δ 10.25 (s, 1H, ar), 8.76 (d, 2H, ar), 8.67 (d, 2H, ar), 8.49 (t, 2H, ar), 8.06 (t, 2H, ar), 5.60 (t, 2H, CH₂), 3.13 (t, 3H, CH₂); ¹³C NMR (125 MHz, (CD₃)₂SO₂) δ 171.40 (C), 151.49 (CH), 140.30 (C), 139.53 (CH), 130.00 (CH), 127.76 (CH), 126.43 (C), 118.42 (CH), 46.16 (CH₂), 32.52 (CH₂); ESI m/z=252.24 (M)+, calc. for C₁₆H₁₄NO₂=252.29

### Target 3: 10-(2-Carboxyethyl)-9-methylacridin-10-ium bromide

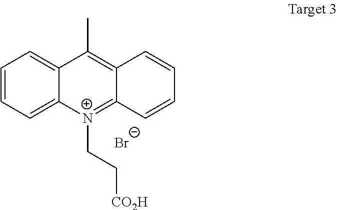 [SMILES: Cc1c2ccccc2[n+](CCC(=O)O)c2ccccc12 ; [Br-]]

Target 3 was obtained in a similar manner to Target 2 but 9-methylacridine was used in place of acridine (Scheme 4).

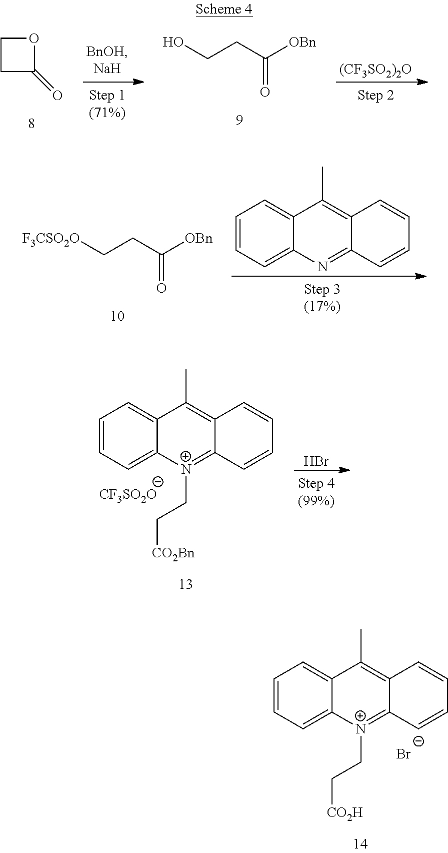

### Step 3

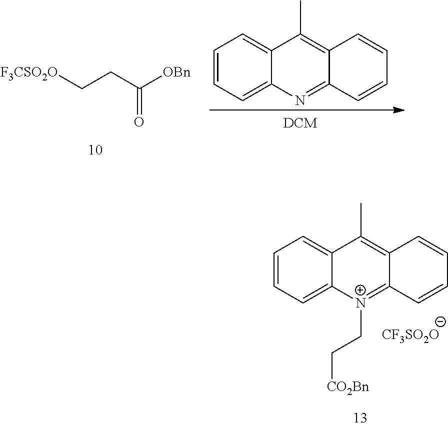

3-Trifluoromethanesulfonyloxy-propionic acid benzyl ester 10 (estimated 3.5 mmol) and 9-methylacridine (386 mg, 2 mmol) were dissolved in DCM (10 mL) and the mixture was stirred at room temperature for 24 h. Concentration in vacuo afforded a black oil and purification by column chromatography (DCM/EtOH, 100-95:0-5) furnished the desired 10-(3-(benzyloxy)-3-oxopropyl)-9-methylacridin-10-ium trifluoromethane sulfonate 13 as a yellow solid (120 mg, 0.232 mmol, 12%). ¹H NMR (500 MHz, CDCl₃) δ 8.69 (d, 2H, ar), 8.60 (d, 2H, ar), 8.32 (t, 2H, ar), 7.92 (t, 2H, ar), 7.27 (m, 5H, ar), 5.69 (t, 2H, CH₂), 5.06 (s, 2H, CH₂), 3.46 (s, 3H, CH₃), 3.27 (t, 3H, CH₂); ¹³C NMR (125 MHz, CDCl₃) δ 169.79 (C), 161.32 (C), 139.93 (CH), 138.51 (C), 135.02 (C), 128.57 (CH), 128.44 (CH), 128.42 (CH), 128.21 (CH), 128.03 (CH), 126.10 (C), 118.52 (CH), 67.53 (CH₂), 46.08 (CH₂), 33.28 (CH₂), 16.25 (CH₃); ESI m/z=356.20 calc. for C₂₄H₂₂NO₂=356.44.

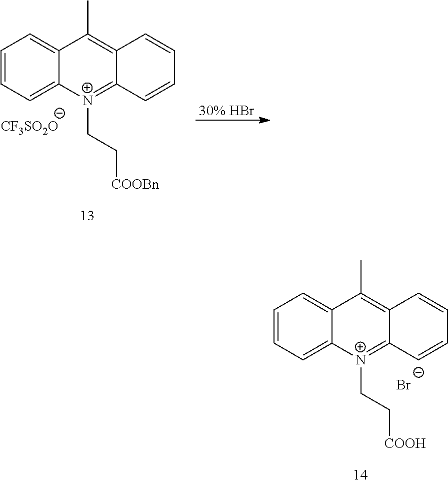

### Step 4

10-(3-(Benzyloxy)-3-oxopropyl)-9-methylacridin-10-ium trifluoromethane sulfonate 13 (100 mg, 0.193 mmol) was dissolved in 30% HBr in acetic acid and the mixture was stirred at 50° C. for 2 h. Concentration in vacuo afforded 10-(2-carboxyethyl)-9-methylacridin-10-ium bromide 14 as a light brown solid (65 mg, 0.187 mmol, 99%). ¹H NMR (500 MHz, (CD₃)₂SO₂) δ 8.93 (d, 2H, ar), 8.72 (d, 2H, ar), 8.44 (t, 2H, ar), 8.03 (t, 2H, ar), 5.54 (t, 2H, CH₂), 3.49 (s, 3H, CH₃), 3.13 (t, 3H, CH₂); ¹³C NMR (125 MHz, (CD₃)₂SO₂) δ 171.41 (C), 161.71 (C), 139.72 (CH), 138.76 (C), 128.32 (CH), 127.40 (CH), 125.67 (C), 118.70 (CH), 46.08 (CH₂), 32.48 (CH₂), 16.59 (CH₃); ESI m/z=266.26 calc. for C₁₇H₁₆NO₂=266.32.

### Derivatisation at the 9-position

### Target 4: 3-(Acridin-9-yl)propionic acid

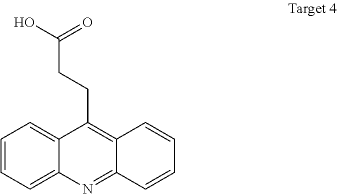 [SMILES: O=C(O)CCc1c2ccccc2nc2ccccc12]

Proposed Synthetic Route—as described in Jensen, H., J. Am. Chem. Soc., 1926, 48, 1988.

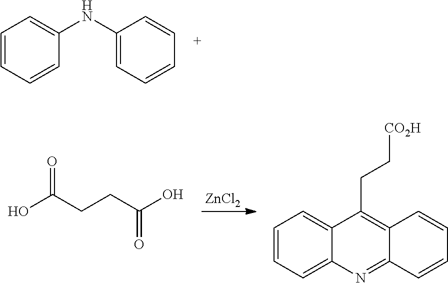

Toma, S. et al., Syn. Commun., 2002, 32, 729

### Target 5: 9-(2-Carboxyethyl)-10-methylacridin-10-ium chloride

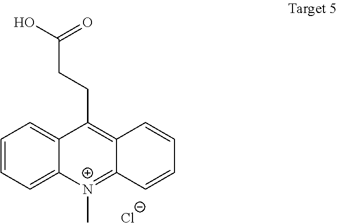 [SMILES: C[n+]1c2ccccc2c(CCC(=O)O)c2ccccc21 ; [Cl-]]

Target 5 has been previously used for the determination of anions in aqueous buffer using fluorescence intensity techniques (Geddes, C. D., Meas. Sci. Technol., 2001, 12, R53; Wolfbeis, O. S., et al., Anal. Chem., 1984, 56, 427; Chen, C.-T. et al., Org. Letters 2009, 11, 4858). However, there are no known reports of Target 5 being used as a fluorescence lifetime reporter.

The synthesis of Target 5 is previously described (Wolfbeis, 0. S., et al., Anal. Chem., 1984, 56, 427), however, a synthetic route was proposed based on our synthetic strategies for the previous targets (Scheme 5).

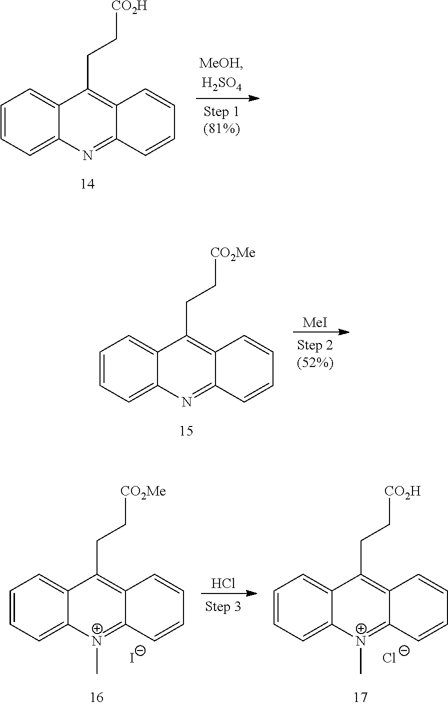

### Step 1

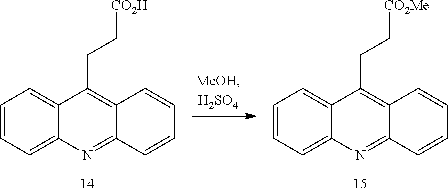 [SMILES: O=C(O)CCc1c2ccccc2nc2ccccc12 ; COC(=O)CCc1c2ccccc2nc2ccccc12]

3-(Acridin-9-yl)propionic acid 14 (200 mg, 0.796 mmol) was dissolved in MeOH (3 mL) and H₂SO₄ (0.5 mL) was added. The mixture was stirred at reflux for 4 h and then was poured into water. The product was extracted with DCM, dried over MgSO₄ and concentrated in vacuo to give methyl 3-(acridin-9-yl)propanoate 15 (170 mg, 0.641 mmol, 81%). Analyses by ¹H NMR, ¹³C NMR and MS conformed to structure.

### Step 2

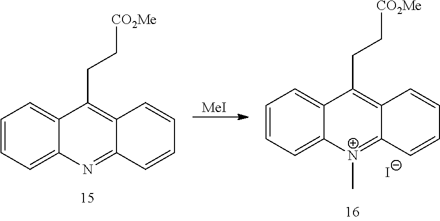 [SMILES: "MeI" ; COC(=O)CCc1c2ccccc2nc2ccccc12 ; COC(=O)CCc1c2ccccc2[n+](C)c2ccccc12 ; [I-]]

Methyl 3-(acridin-9-yl)propanoate 15 (150 mg, 0.565 mmol) was dissolved in Mel (3 mL). The mixture was stirred in a sealed tube at 90° C. for 20 h. The precipitate was collected by filtration and dried under vacuum. Purification by column chromatography (DCM/EtOH, 90:10) afforded 9-(3-methoxy-3-oxopropyl)-10-methylacridin-10-ium iodide 16 (120 mg, 0.295 mmol, 52%). ¹H NMR (500 MHz, CDCl₃) δ 8.89 (d, 2H, ar), 8.71 (d, 2H, ar), 8.43 (t, 2H, ar), 8.00 (t, 2H, ar), 5.15 (s, 3H, NCH₃), 4.27 (t, 2H, CH₂), 3.70 (s, 3H, CH₃), 2.95 (t, 2H, CH₂); ¹³C NMR (125 MHz, (CDCl₃) δ 171.46 (C), 160.91 (C), 141.10 (C), 139.22 (CH), 128.55 (CH), 127.0 (CH), 125.34 (C), 120.25 (CH), 52.45 (CH₃), 41.47 (CH₃), 35.15 (CH₂), 24.91; ESI m/z=280.19 (M)+, calc. for C₁₈H₁₈NO₂=280.28.

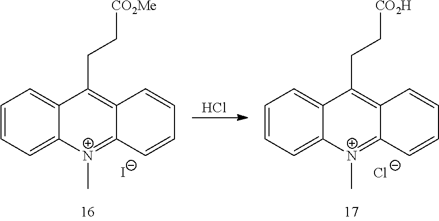 [SMILES: COC(=O)CCc1c2ccccc2[n+](C)c2ccccc12 ; [I-] ; Cl ; C[n+]1c2ccccc2c(CCC(=O)O)c2ccccc21 ; [Cl-]]

### Step 3:

It was anticipated that saponification of the ester to the desired acid could be carried out using the protocol previously described for the synthesis of Target 1 (step 6).

### Example 6

### Synthesis and Fluorescence Properties of 2-[2-(Carbamoylmethyl-carbamoyl)-ethyl]-9,10-dimethyl -acridinium trifluoroacetate salt

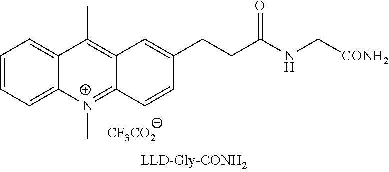

An important feature of a dye functionalised with a group to enable conjugation with a biological molecule such as a peptide is that it is as stable as possible to the conditions used during the conjugation reaction. In this case, the 9,10-dimethylacridin-10-ium methyl acridium core must be stable to the conditions required for activation of the carboxylic acid moiety to enable amide bond formation with an amino group. Consequently, glycine was labelled with 2-(2-carboxyethyl)-9,10-dimethylacridin-10-ium dye on solid support using PyBOP coupling conditions to furnish a target, LLD-Gly-CONH₂ (Scheme 6). This target was selected as it could easily be characterised by ¹H/¹³C NMR and MS to confirm the stability of the dye. Indeed, characterisation by ¹H/¹³C NMR and MS did confirm that the desired target, LLD-Gly-CONH₂, was obtained after reverse phase HPLC purification.

Synthetic Protocol

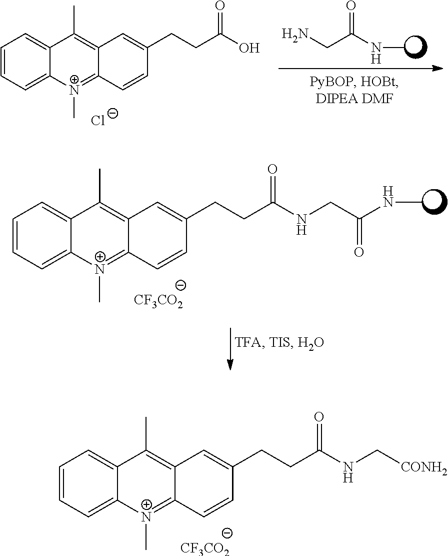

### Scheme 6

2-(2-Carboxyethyl)-9,10-dimethylacridin-10-ium chloride (40 mg, 0.13 mmol) was dissolved in DMF (500 μl) and PyBOP (66 mg, 0.13 mmol), HOBt (917 mg, 0.13 mmol) and DIPEA (88.9 μl, 0.5 mmol) were then added. The mixture was sonicated for 10 minutes. Rink amide bound glycine (200 mg, 0.13 mmol) was allowed to swell in DMF (1 ml) and then the pre-activated dye solution was added to the resin and the mixture sonicated for 4.5 hours. The resin was filtered and washed with DMF and DCM, then dried in vacuo. Water (250 μl), TIS (125 μl) and TFA (5 ml) were added to the dry resin and the mixture was stirred at room temperature for 4 hours. The solution was filtered into cold diethyl ether (30 ml) but no precipitation occurred. The dye-labelled product was extracted into water (2×30 ml) and then lyophilised. Purification by RP-HPLC (Luna C18, 250×4.6 mm column, 10-50% over 40 mins) afforded a dark green fluffy solid (9.75 mg, 23%). ¹H NMR (300 MHz, (CD₃)CN) δ 8.77 (d, J=8.4 Hz, 1H, ar), 8.59 (s, 1H, ar), 8.50 (m, 2H, ar), 8.32 (m, 2H, ar), 7.96 (m, 1H, ar), 4.72 (s, 3H, NCH₃), 3.76 (s, 2H, CH₂), 3.45 (s, 3H, CH₃), 3.28 (t, J=7.5 Hz, 2H, CH₂), 2.77 (t, J=7.5 Hz, 2H, CH₂); ¹³C NMR (100 MHz, CD₃CN) δ 174.04 (C), 173.43 (C), 161.14 (C), 142.11 (C), 141.37 (C), 141.12 (CH), 140.74 (C), 139.02 (CH), 128.97 (CH), 128.37 (CH), 127.20 (CH), 127.15 (C), 127.10 (C), 119.70 (CH), 119.63 (CH), 42.90, (CH₂), 39.39 (CH₃), 37.04 (CH₂), 31.60 (CH₂), 16.36 (CH₃); ESI m/z=336.25 (M)+, calc. for C₂₀H₂₂N₃O₂=336.41; HRMS 336.17060 (M)+, calc. for C₂₀H₂₂N₃O₂=336.17065

### Example 7

### Synthesis of dye labelled peptide using 2-(2-Carboxyethyl)-9,10-dimethylacridin-10-ium chloride—General Protocol

### Step 1: Attachment of 2-(2-Carboxyethyl)-9,10-di methylacridin-10-ium chloride to a peptide

2-(2-Carboxyethyl)-9,10-dimethylacridin-10-ium chloride (22 mg, 0.07 mmol) was dissolved in DMF (200 μl) and PyBOP (36.4 mg, 0.07 mmol), HOBt (9.5 mg, 0.07 mmol) and DIPEA (23 μl, 0.17 mmol) were then added. The mixture was sonicated for 10 minutes. Resin bound peptide (100 mg) was allowed to swell in DMF (500 μl) and then the pre-activated dye solution was added to the resin and the mixture sonicated for 4.5 hours. The resin was filtered and washed with DMF and DCM, then dried in vacuo.

### Step 2: Cleavage of dye-labelled peptide from resin

Water (125μ), TIS (62.5 μl) and TFA (2.5 ml) were added to dry resin (approx 100 mg) and the mixture was stirred at room temperature for 3 hours. The solution was filtered into cold diethyl ether (30 ml) and the precipitated peptide was centrifuged, washed with diethyl ether (30 ml) and lyophilised to give solid. Purification by preparative HPLC furnished the desired product.

### Example 8

### Design of 2-(2-Carboxyethyl)-9,10-dimethylacridin-10-ium labelled Peptide Substrates for Caspase 3 Assay

Two dye labelled peptides, 2-(2-carboxyethyl)-9,10-dimethylacridin-10-ium-DEVDSK (LLD-DEVDSK) and 2-(2-carboxyethyl)-9,10-dimethylacridin-10-ium-DEVDSW (LLD-DEVDSW), were synthesised using the general protocol described in example 7. These peptide substrates were selected for two reasons; (1) to study the effect of tryptophan (W) as a modulator of 2-(2-carboxyethyl)-9,10-dimethylacridin-10-ium fluorescence and (2) DEVDSX is an excellent substrate for the protease Caspase 3.

The fluorescence lifetime of the dye labelled peptides was measured at 1 pM peptide concentration in water and 50 mM TRIS pH 7.5 (Table 3). In 50 mM TRIS pH 7.4, the fluorescence lifetime of LLD-DEVDSK was 29.3 ns. Replacement of the lysine residue with a tryptophan resulted in a significant reduction of the fluorescence lifetime by 21.5 ns to 7.8 ns suggesting that tryptophan is an excellent modulator of the fluorescence lifetime of 9,10-dimethylacridin-10-ium.

TABLE 3
Fluorescence Lifetime Data

| | Average Lifetime (ns) | |
|---|---|---|
| | | 50 mM TRIS pH |
| Peptide Substrate | Water | 7.4 |
| 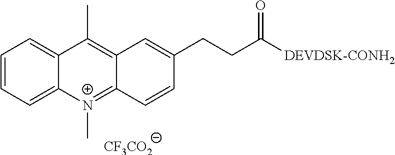 | 31.1 | 29.3 |
| LLD-DEVDSK | | |
| 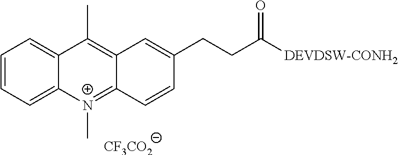 | 7.7 | 7.8 |
| LLD-DEVDSW | | |

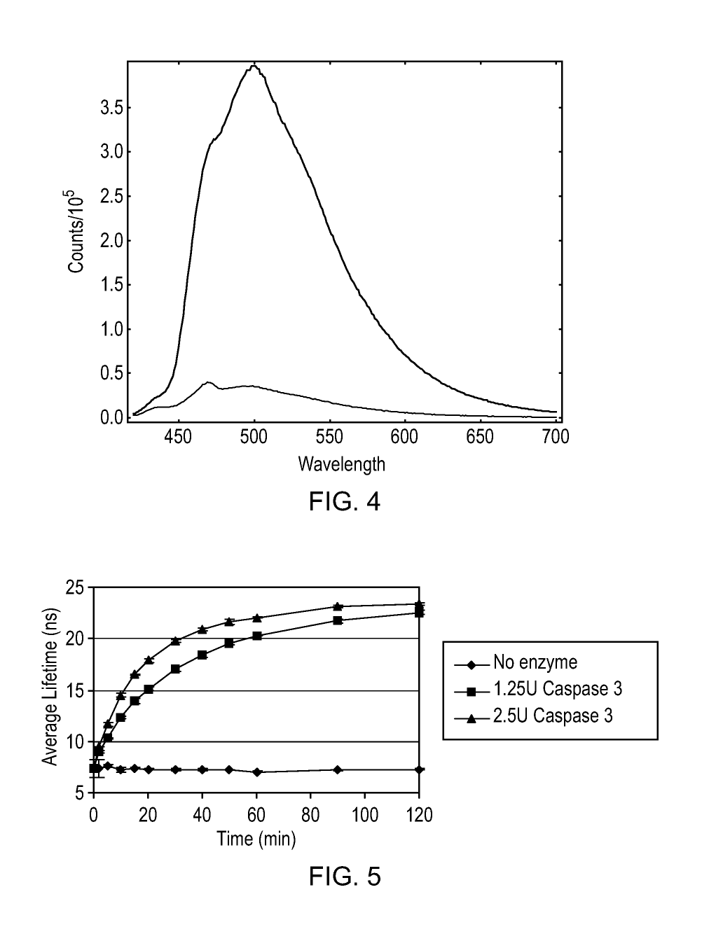

Tryptophan was also shown to modulate the fluorescence intensity of 9,10-dimethylacridin-10-ium. Upon excitation at 405 nm a decrease in fluorescence emission by 90% (at 500 nm) was observed for LLD-DEVDSW compared to LLD-DEVDSK (FIG. 4). Measurements were performed at 500 nM peptide concentration in 10mM PBS.

### Example 9

### Caspase 3 Assay—Using LLD-DEVDSW as Substrate

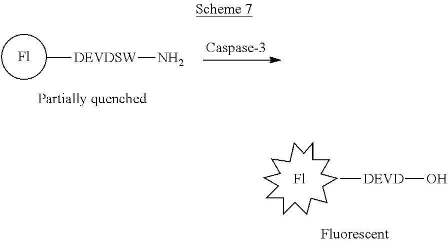

It was anticipated that enzyme mediated cleavage of the tryptophan quenched substrate, LLD-DEVDSW, would produce a fluorescence intensity (increase) and a change in fluorescence lifetime (increase) (Scheme 7), thus enabling the dye to be employed as a fluorescent reporter in biochemical and cell based assays. Thus, the partially quenched 2-(2-carboxyethyl)-9,10-dimethylacridin-10-ium-labelled peptide substrate, LLD-DEVDSW, was employed in a biochemical enzymatic cleavage assay using recombinant Caspase-3 enzyme purchased from R&D Systems. The assay was carried out using 500 nM substrate concentration in 50 mM TRIS buffer pH 7.2 containing 1 mM DTT and 0.1% CHAPS in the presence of either 2.5 or 1.25 units of enzyme (30 μl final volume in a 384 well plate, in triplicate). The assay mixture was analysed in real time, at time intervals, using an Edinburgh Instruments Nanotaurus Fluorescence Lifetime Plate Reader (Ex 405 nm and 473 nm long pass emission filter). During the progress of the reaction a large change in fluorescence lifetime of the reaction mixture was observed (from 7.3 ns to 23.4 ns) indicating that the substrate was being converted to product (FIG. 5). The deviation in fluorescence lifetime from that stated above was a consequence of the buffer and enzyme mixture.

### Example 10

### Caspase 3 Assay—Caspase 3 Inhibition by AcDEVD-CHO

Buffer: 50 mM TRIS pH 7.2 containing 1 mM DTT and 0.1% CHAPS
Inhibitor: AcDEVD-CHO (1000 nM to 0.12 nM, 14 serial dilutions)

### Substrate: LLD-DEVDSW (500 nM)

### Enzyme: Caspase 3 (R&D Systems, 252 U/ml), 2 U/well

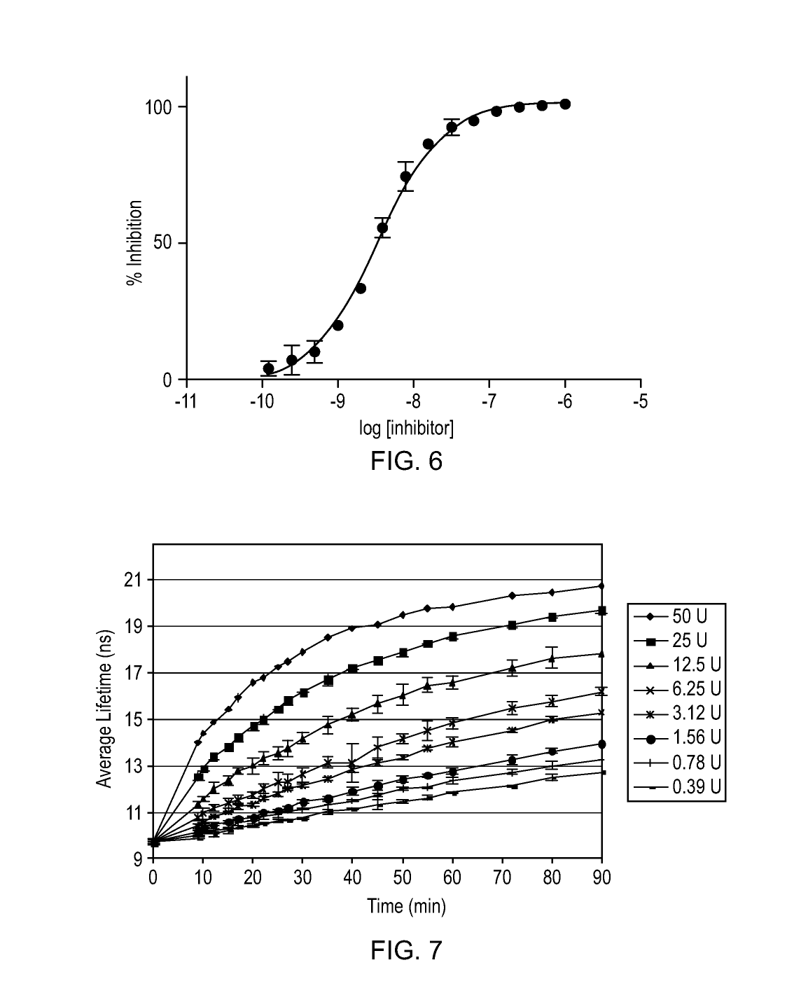

A solution of AcDEVD-CHO (3000 nM in buffer, 3× conc) was serial diluted 2-fold to generate a 14-series inhibitor concentration range. 10 μl of each solution was added to a 384 well plate in triplicate. Enzyme (2 U in 10 μl buffer) was then added to each well and left for 60 minutes. LLD-DEVDSW (10 μl, 1.5 μM in buffer, 3× conc) was added to each well to initiate the assay. The plate was analysed after 20 minutes using an Edinburgh Instruments Nanotaurus Fluorescence Lifetime Plate Reader (Ex 405 nm and 473 nm cut-off emission filter). Plots of average lifetime against log inhibitor concentration were fitted to a variable slope non-linear regression model using GraphPad Prism to give an IC₅₀ value of 3.3 nM for AcDEVD-CHO (see FIG. 6).

### Example 11

### Design of a Substrate for a MMP2 Protease Assay

Following on from the successful use of a 2-(2-carboxyethyl)-9,10-dimethylacridin-10-ium-labelled peptide substrate in the Caspase-3 assay, the same principle was applied to the design of a LLD-labelled peptide substrate for use in a fluorescence lifetime MMP2 protease assay. In this case, the 2-(2-carboxyethyl)-9,10-dimethylacridin-10-ium-labelled peptide substrate, LLD-PLGLNaIAR, contained a naphthylalanine (Nal) residue as the fluorescence modulator, cleavage of which would result in an increase in fluorescence intensity and fluorescence lifetime (Scheme 8).

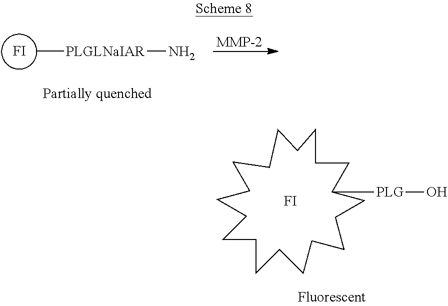

LLD-PLGLNaIAR was synthesised following the general protocol described in Example 7 and employed in a biochemical enzymatic cleavage assay using recombinant MMP2 enzyme purchased from EnzoLifeSciences. The assay was carried out using 1 μM substrate concentration in 50 mM TRIS buffer pH 7.5 containing 150 mM NaCl, 1 mM CaCl₂, 1 mM ZnCl₂ and 0.1% CHAPS in the presence of varying enzyme concentration (30 μl final volume in a 384 well plate, in triplicate). The assay mixture was analysed in real time, at time intervals, using an Edinburgh Instruments Nanotaurus Fluorescence Lifetime Plate Reader (Ex 405 nm and 473 nm long pass emission filter). During the progress of the reaction a large change in fluorescence lifetime of the reaction mixture was observed (from 9.7 ns to 20.8 ns) indicating that the substrate was being converted to product (FIG. 7).

### Example 12

### Design of a Substrate for a Tyrosine Kinase Assay

The following dye labelled peptides were synthesised using the protocol described in Example 7; LLD-EPEGIYGVLF (Lck substrate), LLD-EPEGIpYGVLF (Lck Product), LLD-GGEEEEYFELVKK (Jak2/3 substrate) and LLD-GGEEEEpYFELVKK (Jak2/3 product). These peptides were selected for two reasons; (1) to study the effect of tyrosine (Y) as a modulator of 2-(2-carboxyethyl)-9,10-dimethylacridin-10-ium fluorescence and (2) EPEGIYGVLF is a substrate for Lck kinase, and GGEEEEYFELVKK a substrate for Jak2 and Jak3 kinases.

The fluorescence lifetime of the dye labelled peptides was measured at 1 μM peptide concentration in 50 mM TRIS pH 7.4 containing 40 μM ATP, 10 mM MgCl₂ and 1 mg/ml BSA (Table 4). An increase of 5.2 ns (from 13.0 ns to 18.2 ns) was observed between non-phosphorylated and phosphorylated Lck peptides, and an increase of 4.8 ns (from 11.3 ns to 16.1 ns) between non-phosphorylated and phosphorylated Jak2/3 peptides suggesting that tyrosine is also a good modulator of the fluorescence lifetime of 9,10-dimethylacridin-10-ium. Upon phosphorylation, the neutral phenol moiety is converted into a negatively charged species thereby alleviating some of the modulating effects of tyrosine and consequently an increase in fluorescence is observed. The modulating effect caused by tyrosine is only partially alleviated upon phosphorylation and hence the fluorescence lifetime of phosphorylated peptide is not as long as for the free dye.

TABLE 4
Fluorescence Lifetime of Tyrosine Kinase Peptides

| Peptide Substrate | Average Lifetime (ns) |
|---|---|
| 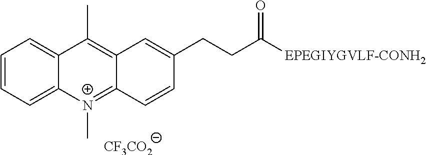 | 13.0 |
| LLD-EPEGIYGVLF | |
| 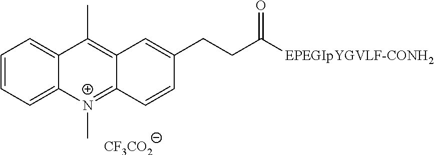 | 18.2 |
| LLD-EPEGIpYGVLF | |
| 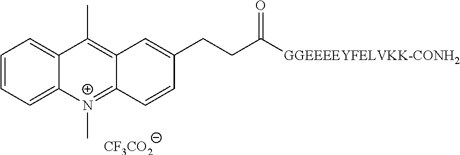 | 11.3 |
| LLD-GGEEEEYFELVKK | |
| 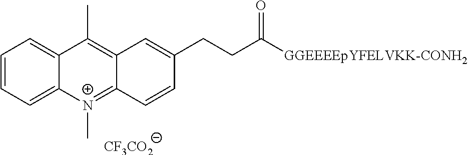 | 16.1 |
| LLD-GGEEEEpYFELVKK | |

### Example 13

### Lck Tyrosine Kinase Assay—Using LLD-EPEGIYGVLF Substrate

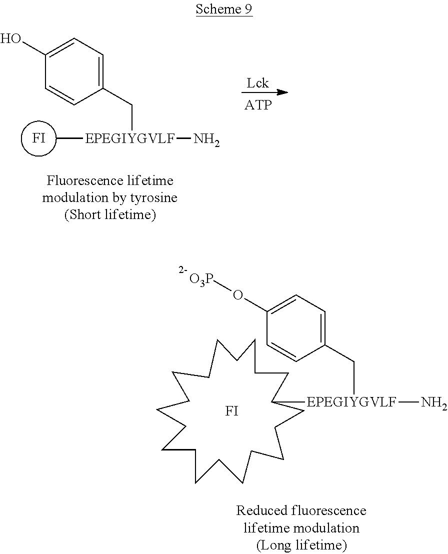

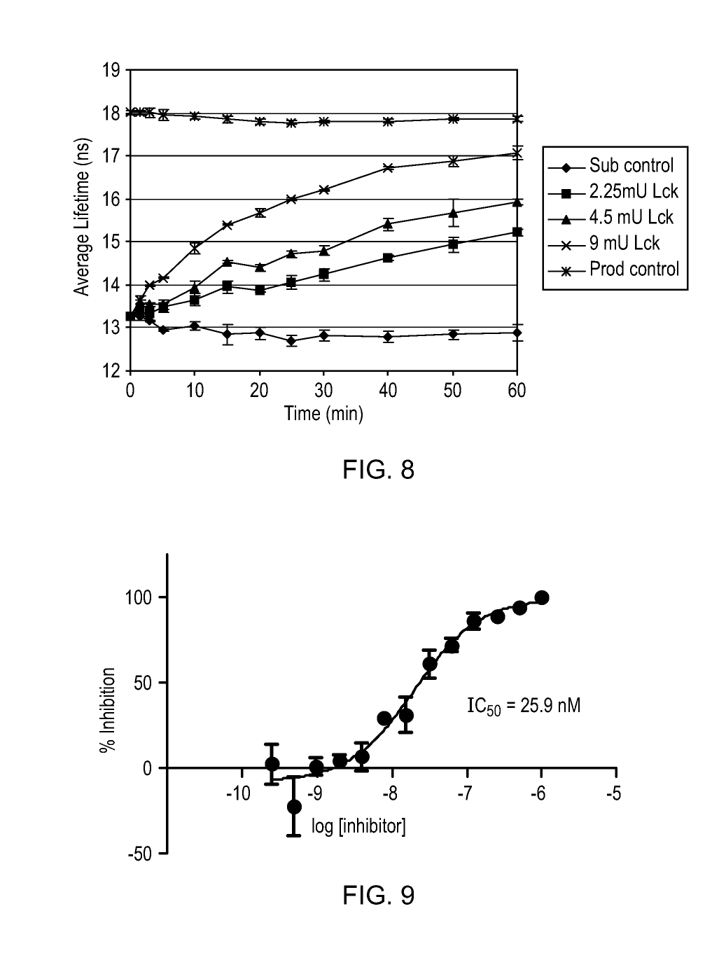

It was proposed that kinase mediated phosphorylation of LLD-labelled substrate in the presence of ATP would result in an increase in fluorescence lifetime and fluorescence intensity that could be monitored in real-time. Thus, the partially quenched 2-(2-carboxyethyl)-9,10-dimethylacridin-10-ium-labelled peptide substrate, LLD-EPEGIYGVLF, was employed in a biochemical enzymatic phosphorylation assay using recombinant Lck (p56lck) enzyme purchased from EnzoLifeSciences. The assay was carried out using 1 μM substrate concentration in 50 mM TRIS buffer pH 7.2 containing 40 μM ATP, 10 mM MgCl₂ and 1 mg/ml BSA in the presence of varying enzyme concentration (30 μl final volume in a 384 well plate, in triplicate). The assay mixture was analysed in real time, at time intervals, using an Edinburgh Instruments Nanotaurus Fluorescence Lifetime Plate Reader (Ex 405 nm and 473 nm long pass emission filter). During the progress of the reaction a change in fluorescence lifetime of the reaction mixture was observed (from 13.2 ns to 17.1 ns) indicating that the substrate was being converted to product (FIG. 8).

### Example 14

### Lck Tyrosine Kinase Assay—Lck Inhibition by Staurosporine

Buffer A: 50 mM TRIS pH 7.2 containing 1 mg/ml BSA
Buffer B: 50 mM TRIS pH 7.2 containing 80 μM ATP, 20 mM MgCl₂ and 1 mg/ml BSA
Inhibitor: Staurosporine (1000 nM to 0.12 nM, 14 serial dilutions)

### Substrate: LLD-EPEGIYGVLF (1 μM)

Enzyme: Lck (p56lck) (EnzoLifeSciences, 18 U/ml), 4.5 mU/well

A solution of staurosporine (6000 nM in buffer A, 6× conc) was serial diluted 2-fold to generate a 14-series inhibitor concentration range. 5 μl of each solution was added to a 384 well plate in triplicate. Enzyme (4.5 mU in 10 μl buffer A) was then added to each well and left for 15 minutes. LLD-EPEGIYGVLF (15 μl, 2 μM in buffer B, 2× conc) was added to each well to initiate the assay. The plate was analysed after 30 minutes using an Edinburgh Instruments Nanotaurus Fluorescence Lifetime Plate Reader (Ex 405 nm and 473 nm cut-off emission filter). Plots of percentage inhibition against log inhibitor concentration were fitted to a variable slope non-linear regression model using GraphPad Prism to give an IC₅₀ value of 25.9 nM for staurosporine (see FIG. 9).

### Example 15

### Lck Tyrosine Kinase Assay—ATP Km Determination

Buffer A: 50 mM TRIS pH 7.2 containing 1 mg/ml BSA
Buffer B: 50 mM TRIS pH 7.2 containing 80 μM ATP, 20 mM MgCl₂ and 1 mg/ml BSA
ATP: 200 μM to 98 nM, 12 serial dilutions

### Substrate: LLD-EPEGIYGVLF (1 μM)

Enzyme: Lck (p56Ick) (EnzoLifeSciences, 18 U/m1), 3.6 mU/well

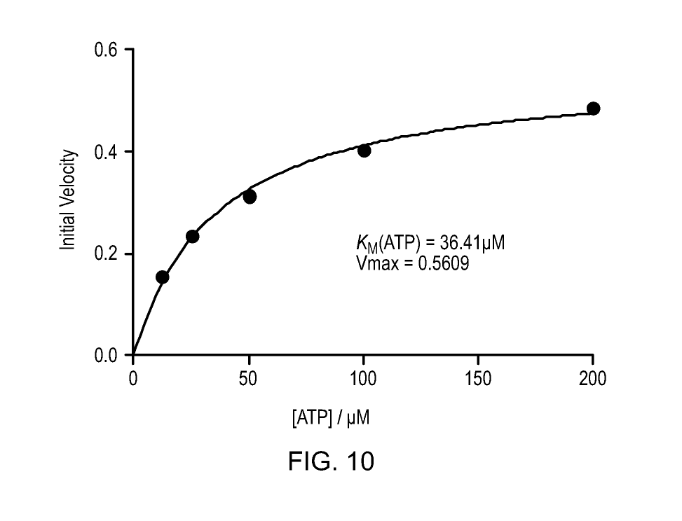

A solution of ATP (600 μM in buffer A, 3× conc) was serial diluted 2-fold to generate a 14-series concentration range. 10 μl of each solution was added to a 384 well plate in triplicate. Enzyme (3.6 mU in 5 μl buffer A) was then added to each well. LLD-EPEGIYGVLF (15 μl, 2 μM in buffer B, 2× conc) was added to each well to initiate the assay. The plate was analysed in real time, at time intervals, using an Edinburgh Instruments Nanotaurus Fluorescence Lifetime Plate Reader (Ex 405 nm and 473 nm cut-off emission filter). The average lifetime values were converted to pmol phosphopeptide from a standard curve and plots of initial rates of reaction dependent on ATP concentration were used to determine the ATP Km value by non-linear regression fitting using GraphPad Prism (see FIG. 10). The ATP Km was determined to be 36.41 μM for Lck kinase using LLD-EPEGIYGVLF.

### Example 16

### Attachment of Dye Derivatives III, IV, V, VI and VII to a Peptide for Fluorescence Lifetime Analysis

Using the general protocol described in Example 7 a peptide substrate, DEVDSK, was labelled via the N-terminus with each of the dye derivatives (III-VII) to enable the fluorescence lifetime values of each derivative to be measured and compared. The fluorescence lifetime of the dye labelled peptides was measured at 1 μM concentration in water and 50 mM TRIS pH 7.4 (Table 5).

TABLE 5
Comparison of Long Lifetime Dye Derivatives

| | | Average Lifetime (ns) | |
|---|---|---|---|
| Dye | | | 50 mM TRIS |
| Derivative | Peptide Substrate | Water | pH 7.4 |
| (III) | 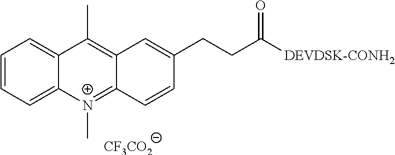 | 31.1 | 29.3 |
| (IV) | 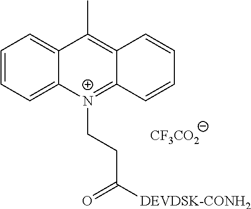 | 26.7 | 25.2 |
| (v) | 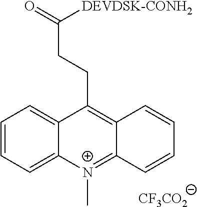 | 27.0 | 25.0 |
| (VI) | 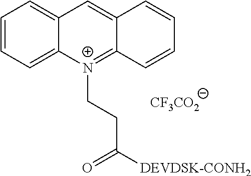 | 24.1 | 19.1 |
| (VII) | 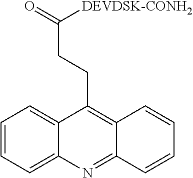 | 23.6 | 17.2 |

1. A method comprising measuring the fluorescence lifetime of a conjugate of a compound of formula (I): 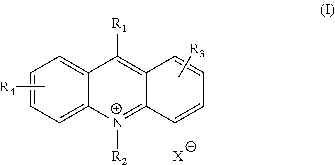
(wherein:
R¹ is hydrogen or J-L;
R² is absent, hydrogen or J-L;
R³ and R⁴ are independently at each occurrence selected from hydrogen, halo, amido, hydroxyl, substituted or unsubstituted alkyl, substituted or unsubstituted alkenyl, substituted or unsubstituted aryl, alkoxy, alkylthio, amino, mono- or di-C₁-C₄ alkyl-substituted amino, sulfhydryl, carboxy, acyl, formyl, sulfonate, quaternary ammonium, J-L, or —K;
X is absent if R² is absent, and if R² is present the nitrogen atom to which it is attached is positively charged and X is a counter ion;
each J is independently a linker group;
each L is independently hydrogen or K; and
each K is independently a target bonding group, provided that at least one group K is present) and a target molecule.

2. The method of claim 1 wherein each J is independently comprises from 1 to 40 chain atoms comprising carbon, and optionally nitrogen, oxygen, sulfur and/or phosphorus.

3. The method of claim 2 wherein each J is independently a substituted or unsubstituted alkylene, alkenylene, alkyleneoxy chain or an alkyleneanecarboxamido chain.

4. The method of claim 3 wherein each J is independently an unsubstituted alkylene chain comprising 2 to 6 carbon atoms.

5. The method of claim 1 wherein each K is independently selected from the group consisting of succinimidyl ester, sulpho-succinimidyl ester, isothiocyanate, maleimide, haloacetamide, acid halide, vinylsulfone, dichlorotriazine, carbodimide, hydrazide, phosphoramidite pentafluoro phenyl ester, and alkyl halide, hydroxy, amino, sulfhydryl, imidazole, carboxyl, carbonyl, phosphate, thiophosphate and aminooxy.

6. The method of claim 1, wherein one group K is present.

7. The method of claim 6 wherein the group K is carboxyl.

8. The method of claim 1 wherein:
(i) group R₂ is present and is of formula -J-L; and/or
(ii) group R₁ is hydrogen or of formula -J-L.

9. The method of claim 8 wherein:
(i) group R₂ is either an unsubstituted alkyl group or of formula -J-K; and/or
(ii) group R₁ is of formula -J-K.

10. The method of claim 8 wherein R¹ is hydrogen or J-H; R² is J-L; and R³ is hydrogen or J-K.

11. The method of claim 1 wherein R₂ and counter ion X are present.

12. The method of claim 1 wherein the compound has one of the formulae (III), (IV), (V), (VI) or (VII):

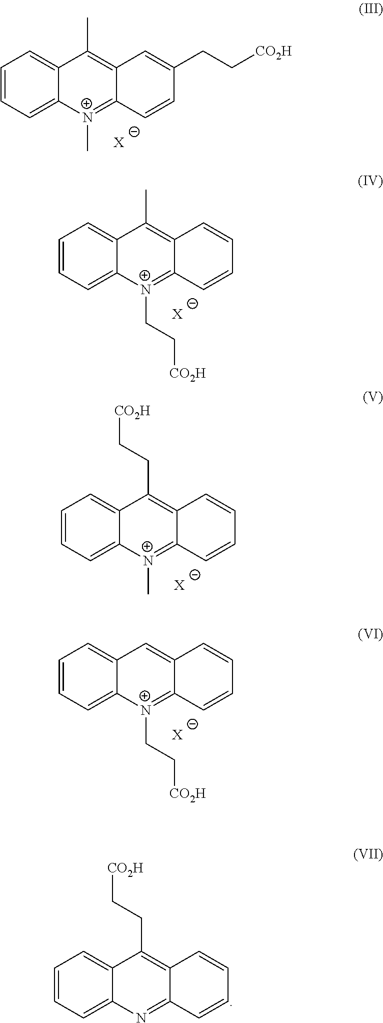

13. The method of claim 1 wherein the target molecule is a biological molecule.

14. The method of claim 13 wherein the biological molecule is selected from the group consisting of antibodies, lipids, proteins, peptides, carbohydrates, nucleotides and oxy or deoxy polynucleic acids, which contain or are derivatised to contain one or more of an amino, sulphydryl, carbonyl, hydroxyl and carboxyl, phosphate, thiophosphate aminooxy and hydrazide group.

15. A method for determining the presence of an analyte in a sample, which method comprises:
(i) contacting the sample with a conjugate of a known binding partner of the analyte and a fluorescent dye of the formula (I): 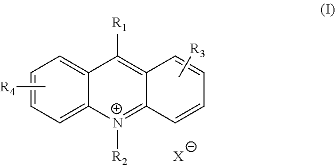
(wherein:
R¹ is hydrogen or J-L;
R² is absent, hydrogen or J-L;
R³ and R⁴ are independently at each occurrence selected from hydrogen, halo, amido, hydroxyl, substituted or unsubstituted alkyl, substituted or unsubstituted alkenyl, substituted or unsubstituted aryl, alkoxy, alkylthio, amino, mono- or di-C₁-C₄ alkyl-substituted amino, sulfhydryl, carboxy, acyl, formyl, sulfonate, quaternary ammonium, J-L, or —K;
X is a counter ion, which is absent if R² is absent;
each J is independently a linker group;
each L is independently hydrogen or K; and
each K is independently a target bonding group, provided that at least one group K is present) under conditions effective to allow binding of at least a portion of the analyte to the known binding partner within the conjugate to form a complex of the analyte and the conjugate;
(ii) measuring the fluorescence lifetime or fluorescence intensity of the conjugate before contact with the analyte; and
(iv) measuring the fluorescent lifetime or fluorescence intensity of the mixture resultant from the contacting.

16. The method of claim 15 wherein the analyte and known binding partner are selected from the group consisting of antibodies/antigens, lectins/glycoproteins, biotin/streptavidin, hormone/receptor, enzyme/substrate or co-factor, DNA/DNA, DNA/RNA and DNA/binding protein.

17. The method of claim 15 wherein the analyte is an enzyme and the known binding partner a substrate or cofactor therefor; or the analyte is a substrate or cofactor for an enzyme, which enzyme is the known binding partner.

18. A method of measuring activity of an enzyme in the presence of a conjugate, which is a conjugate resultant from conjugation between a compound and a fluorescent dye of the formula (I): 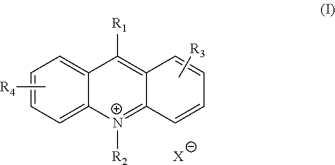
(wherein:
R¹ is hydrogen or J-L;
R² is absent, hydrogen or J-L;
R³ and R⁴ are independently at each occurrence selected from hydrogen, halo, amido, hydroxyl, substituted or unsubstituted alkyl, substituted or unsubstituted alkenyl, substituted or unsubstituted aryl, alkoxy, alkylthio, amino, mono- or di-C₁-C₄ alkyl-substituted amino, sulfhydryl, acyl, formyl, carboxy, sulfonate, quaternary ammonium, J-L, or —K;
X is a counter ion, which is absent if R² is absent;
each J is independently a linker group;
each L is independently hydrogen or K; and
each K is independently a target bonding group, provided that at least one group K is present), which method comprises:
(i) measuring the fluorescence lifetime or fluorescence intensity of the conjugate before contact with the enzyme;
(ii) contacting the enzyme with the conjugate; and
(iii) measuring the fluorescent lifetime or fluorescence intensity of the mixture resultant from the contacting.

19. The method of claim 18 wherein the compound of the conjugate is a biological molecule.

20. The method of claim 18 wherein the enzyme is selected from the group consisting of kinases, phosphatases, proteases, esterase, peptidases, amidases, nucleases and glycosidases.

21. The method of claim 19 wherein the biological molecule is a substrate for the enzyme and is capable of being cleaved by it.

22. The method of claim 21 wherein the substrate comprises a fluorescence-modulating moiety which moiety is separated from the fluorescent dye of formula (I) upon cleavage of the substrate by the enzyme.

23. The method of claim 22 wherein the fluorescence-modulating moiety is a tryptophan, tyrosine, histidine, naphthylalanine or phenylalanine residue or a naphthyl, indolyl or phenoxy group.

24. The method of claim 19 wherein the enzyme is a kinase and the biological molecule is a substrate for the kinase.

25. The method of claim 24 wherein the substrate comprises a fluorescence-modulating moiety which is altered upon phosphorylation of the substrate by the kinase.

26. The method of claim 24, which is carried out in the presence of fluorescence-modulating moieties constituted by polydentate ligands coordinated to iron (III) ions and phosphorylation of the substrate by the kinase provides phosphate groups to which the iron (III) ions coordinate.

27. The method of claim 18 wherein the method is carried out in both the presence and in the absence of a test compound.

28. The method of claim 15 wherein each measuring is measuring of fluorescence lifetime.

29. (canceled)

29. A fluorescent dye of formula (I) as defined in claim 10.

30. A conjugate of a fluorescent dye of formula (I) as defined in claim 10 and a biological molecule.

31. A kit comprising:
(i) a conjugate of a fluorescent dye of formula (I) as defined in claim 10 and a biological molecule; and
(ii) a known binding partner of the biological molecule.

32. The kit of claim 31 wherein the known binding partner is an enzyme and the biological molecule is a substrate or cofactor for the enzyme.

33. The method of claim 18 wherein each measuring is measuring of fluorescence lifetime.
